(19) United States
(12) Patent Application Publication
(10) Pub. No.: US 2023/0417766 A1
(43) Pub. Date: Dec. 28, 2023

(54) METHODS FOR DECREASING MORTALITY RISK AND IMPROVING HEALTH

(71) Applicant: AMGEN INC., Thousand Oaks, CA (US)

(72) Inventors: Kári Stefánsson, Reykjavík (IS); Daniel Gudbjartsson, Reykjavík (IS); Magnús Úlfarsson, Reykjavík (IS); Thjodbjorg Eiriksdottir, Reykjavík (IS)

(21) Appl. No.: 18/035,016

(22) PCT Filed: Nov. 15, 2021
(86) PCT No.: PCT/US21/59389

§ 371 Date: May 2, 2023

(51) Int. Cl. G01N 33/68; A61K 38/17; C07K 16/40; C07K 16/24; A61P 11/06; A61P 19/02; A61P 3/06; C07K 16/28

(52) U.S. Cl. CPC G01N 33/6893; A61K 38/1793; C07K 16/40; C07K 16/244; A61P 11/06; A61P 19/02; A61P 3/06; C07K 16/2875; G01N 2800/52; C07K 2317/21; A61K 2039/505

(57) ABSTRACT

The disclosure provides methods for decreasing mortality risk and/or improving health in a subject. In some aspects, the disclosure provides methods of using one or more biomarkers to determine the risk of mortality, detect an increase or decrease over time in the risk of mortality, or detect an improvement or decline in health of a subject that occurs, for example, over time, in response to therapeutic intervention, or as a result of changes in diet, fitness, or other lifestyle changes. In some other aspects, the disclosure provides methods for treating a subject to reduce the mortality risk of the subject. Additionally, the disclosure provides methods for using changes in the risk of mortality of a subject to monitor the effectiveness of a therapeutic treatment, dietary restrictions, fitness regimen, or other intervention in a subject, and for continuing, discontinuing, or altering the treatment, restrictions, regimen, or intervention accordingly. Further, the disclosure provides methods for predicting effectiveness of a therapeutic treatment in a subject determined to be at an increased risk of mortality and subsequently treating the subject with the therapeutic treatment if the biomarker level is indicative of effectiveness of the treatment of the subject.

### FIELD

The disclosure generally relates to methods for decreasing mortality risk and/or improving health in a subject. In some aspects, the disclosure provides methods of using one or more biomarkers to determine the risk of mortality, detect an increase or decrease over time in the risk of mortality, or detect an improvement or decline in health of a subject that occurs, for example, over time, in response to therapeutic intervention, or as a result of changes in diet, fitness, or other lifestyle changes. In some other aspects, the disclosure provides methods for treating a subject to reduce the mortality risk of the subject. Additionally, the disclosure provides methods for using changes in the risk of mortality of a subject to monitor the effectiveness of a therapeutic treatment, dietary restrictions, fitness regimen, or other intervention in a subject, and for continuing, discontinuing, or altering the treatment, restrictions, regimen, or intervention accordingly. Further, the disclosure provides methods for predicting effectiveness of a therapeutic treatment in a subject determined to be at an increased risk of mortality and subsequently treating the subject with the therapeutic treatment if the biomarker level is indicative of effectiveness of the treatment of the subject.

### BACKGROUND

The ability to measure an improvement in health, a decline in health or an increased or decreased risk of poor health or mortality could prove useful in the delivery of healthcare and biomedical research. It is often possible to give a meaningful prediction of how long individuals with specific diagnoses will live, but predicting when an individual will die from any cause is altogether a different matter.

Several diseases and lifestyle (Hippisley-Cox, J. & Coupland, C. Development and validation of QMortality risk prediction algorithm to estimate short term risk of death and assess frailty: cohort study. BMJ 358, (2017); Flegal, K. M., Kit, B. K., Orpana, H. & Graubard, B. I. Association of All-Cause Mortality With Overweight and Obesity Using Standard Body Mass Index Categories: A Systematic Review and Meta-analysis. JAMA 309, 71-82 (2013); Danaei, G. et al. The Preventable Causes of Death in the United States: Comparative Risk Assessment of Dietary, Lifestyle, and Metabolic Risk Factors. PLoS Med. 6, (2009)), social, and psychological factors (Puterman, E. et al. Predicting mortality from 57 economic, behavioral, social, and psychological factors. Proc. Natl. Acad. Sci. (2020) doi:10.1073/pnas.1918455117) associate with all-cause mortality. Commonly used risk factors for all-cause mortality are age, sex, traditional cardiovascular risk factors such as systolic blood pressure, cholesterol levels, smoking, diabetes, and cardiovascular disease, cancer, alcohol consumption, body mass index (BMI), and creatinine levels (Wang, T. J. et al. Multiple Biomarkers for the Prediction of First Major Cardiovascular Events and Death. N. Engl. J. Med. 9 (2006); Fischer, K. et al. Biomarker Profiling by Nuclear Magnetic Resonance Spectroscopy for the Prediction of All-Cause Mortality: An Observational Study of 17,345 Persons. PLoS Med. 11, e1001606 (2014); Deelen, J. et al. A metabolic profile of all-cause mortality risk identified in an observational study of 44,168 individuals. Nat. Commun. 10, 3346 (2019)). Among other biomarkers of all-cause mortality are brain age obtained from structural neural images (Cole, J. H. et al. Brain age predicts mortality. Mol. Psychiatry 23, 1385-1392 (2018)), DNA methylation (Zhang, Y. et al. DNA methylation signatures in peripheral blood strongly predict all-cause mortality. Nat. Commun. 8, 14617 (2017)), and telomere length (Cawthon, R. M., Smith, K. R., O'Brien, E., Sivatchenko, A. & Kerber, R. A. Association between telomere length in blood and mortality in people aged 60 years or older. The Lancet 361, 393-395 (2003)). Recently, circulating metabolomic biomarkers have been screened to search for biomarkers that associate with the risk of all-cause mortality. In a study of 44,168 individuals, where 5,512 died during follow-up, 14 metabolomic biomarkers were found to improve five and ten-year all-cause mortality prediction over conventional risk factors (Deelen et al., supra). Another study of 17,345 participants identified 106 metabolic biomarkers that improved short-term all-cause mortality risk prediction over established risk factors (Fischer et al., supra). In a study of 3,523 participants from the Framingham Heart Study, 38 of 85 preselected circulating protein biomarkers were associated with all-cause mortality and found to improve all-cause mortality prediction over cardiovascular risk factors (Ho, J. E. et al. Protein biomarkers of cardiovascular disease and mortality in the community. J. Am. Heart Assoc. 7, (2018)).

With the advent of new technology such as SOMAmers (Rohloff, J. C. et al. Nucleic Acid Ligands With Protein-like Side Chains: Modified Aptamers and Their Use as Diagnostic and Therapeutic Agents. Mol. Ther. Nucleic Acids 3, e201 (2014)) or proximity extension assays (Assarsson, E. et al. Homogenous 96-Plex PEA Immunoassay Exhibiting High Sensitivity, Specificity, and Excellent Scalability. PLOS ONE 9, e95192 (2014)), it is now possible to simultaneously measure thousands of proteins efficiently. This technology has been used to create a protein-based risk score for cardiovascular outcomes in a high-risk group using 1,130 candidate plasma proteins (Ganz, P. et al. Development and Validation of a Protein-Based Risk Score for Cardiovascular Outcomes Among Patients With Stable Coronary Heart Disease. JAMA 315, 2532-2541 (2016)). Another study of 4,263 participants measured 2,925 proteins to evaluate how circulating protein profile changes over the lifespan (Lehallier, B. et al. Undulating changes in human plasma proteome profiles across the lifespan. Nat. Med. 25, 1843-1850 (2019)). In addition, ˜5,000 plasma proteins were used to predict health states, behavior, and incident diseases in 16,894 participants (Williams, S. A. et al. Plasma protein patterns as comprehensive indicators of health. Nat. Med. 25, 1851-1857 (2019)). These studies underscore the value of using plasma levels of a large number of proteins to search for biomarkers in health and diseases.

However, the art to date does not disclose robust methods for detecting an improvement or a decline in health, an increased or decreased risk of mortality, or predicting the probability of declining health or mortality using one or more biomarkers. Accordingly, a strong need in the art exists for a reliable way of using biomarkers to determine the risk of mortality of a subject and to detect an improvement or decline in health of a subject, including an improvement or decline in health resulting from a therapeutic or other intervention, or to detect an increased or decreased risk in mortality over time. The following disclosure specifically describes such biomarkers and their uses.

### SUMMARY

The invention described herein provides compositions and methods for determining the mortality risk of a subject. Methods also are provided for decreasing the risk of mortality and/or improving the health of a subject. These compositions and methods can be used beneficially in a wide variety of contexts and in a wide variety of subjects. Non-limiting examples of such uses include in subjects with any of a wide range of disease states or indications, or who are being treated or considered for treatment with any of a wide range of therapeutic interventions, with or at risk for age-related declines in health, with or at risk of developing obesity, cachexia, or other metabolic condition, or who have or are at risk of developing other physiological, psychological, or behavioral conditions. In some aspects, the disclosure provides methods of using one or more biomarkers to measure an increased or decreased risk of mortality of a subject or measure an improvement or decline in health of the subject. More particularly, the disclosure provides methods for determining the risk of mortality of a subject, once or repeatedly over a period of time, by measuring the level of a biomarker or a combination of biomarkers in a biological sample and using that information to initiate, alter, or terminate a therapeutic intervention for the subject. The disclosure provides new biomarkers and combinations of biomarkers used in methods for measuring risk of mortality of a subject, methods for treating a subject determined to be at an increased risk of mortality, and methods for monitoring the effectiveness of a therapeutic treatment in reducing the risk of mortality of the subject.

The disclosure provides a method of treating a subject to reduce risk of mortality of the subject, the method comprising:
measuring the level of a protein in a biological sample from the subject;
comparing the measured level of the protein to a reference level for the protein, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2); and
administering to the subject an effective dose of a therapeutic treatment to decrease the risk of mortality of the subject if
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level and/or;
   (ii) the level of ANTRX2, HPGDS, ADAM22, GHR, or CILP2 is decreased relative to the reference level.

The disclosure provides a method for determining the efficacy of a therapeutic treatment in a subject to reduce risk of mortality of a subject, the method comprising: measuring the level of a protein in a biological sample from the subject before the therapeutic treatment, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2);
measuring the level of the protein in a biological sample from the subject after administering the treatment; and
determining that the treatment is effective in reducing risk of mortality of the subject if
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is decreased relative to the reference level or to the level in the sample from the subject before treatment; and/or
   (ii) the level of ANTRX2, HPGDS, ADAM22, GHR, or CILP2 is increased relative to the reference level or to the level in the sample from the subject before treatment.

In some aspects, the methods comprise measuring the level of each of any two or more of
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; and/or
(at) WNT1-inducible-signaling pathway protein 2 (WISP-2); and
comparing the measured level of each of the proteins to the reference level for each of the proteins.

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) anthrax toxin receptor 2 (ANTRX2).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-2 (SPON2);
(h) serum amyloid A-1 protein (SAA1);
(i) serum amyloid A-2 protein (SAA2); and/or
(j) C5a anaphylatoxin (C5aAT).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSFiA);
(h) angiopoietin-2 (ANG2);
(i) macrophage metalloelastase (MMP12); and/or
(j) spondin-2 (SPON2).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSF1A);
(h) angiopoietin-2 (ANG2);
(i) macrophage metalloelastase (MMP12); and/or
(j) transgelin (TAGLN).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) angiopoietin-2 (ANG2);
(g) tumor necrosis factor receptor superfamily member 1A (TNFRSF1A);
(h) macrophage metalloelastase (MMP12);
(i) sushi, von Willebrand factor type A, EGF and pentraxin domain-containing protein 1 (SVEP1); or
(j) C5a anaphylatoxin (C5aAT).

In some aspects, the protein and/or proteins is
(a) growth/differentiation factor 15 (GDF15);
(b) thrombospondin-2;
(c) retinoblastoma-like protein 2;
(d) WAP four-disulfide core domain protein 2;
(e) anthrax toxin receptor 2;
(f) alpha-1-antichymotrypsin complex;
(g) spondin-2;
(h) serum amyloid A-1;
(i) serum amyloid A-2;
(j) C5a anaphylatoxin;
(k) tumor necrosis factor receptor superfamily member 1A;
(l) angiopoietin-2;
(m) macrophage metalloelastase;
(n) transgelin; and/or
(o) sushi, von Willebrand factor type A, EGF and pentraxin domain-containing protein 1.

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) alpha-1-antichymotrypsin complex (ACT COMPLEX or SERPINA3).

In some aspects, the protein and/or proteins is
(a) hematopoietic prostaglandin D synthase (HPGDS);
(b) anthrax toxin receptor 2 (ANTRX2);
(c) disintegrin and metalloproteinase domain-containing protein 22 (ADAM22); (d) growth hormone receptor (GHR); and/or
(e) insulin-like growth factor-binding protein 2 (IGFPB2).

In some aspects, the protein and/or proteins is
(a) growth/differentiation factor 15 (GDF15);
(b) WAP four-disulfide core domain protein 2 (WFDC2);
(c) cardiac troponin T (cTnT);
(d) cartilage intermediate layer protein 2 (CILP2); and/or
(e) hematopoietic prostaglandin D synthase (HPGDS).

In some aspects, the protein and/or proteins is
(a) growth/differentiation factor 15 (GDF15);
(b) WAP four-disulfide core domain protein 2 (WFDC2);
(c)N-terminal pro-BNP (NTproBNP);
(d) transmembrane emp24 domain-containing protein 10 (TMED10); and/or
(e) pancreatic ribonuclease (RNase1).

In some aspects, the protein and/or proteins is
(a) growth/differentiation factor 15 (GDF15);
(b) tyrosine-protein kinase transmembrane receptor ROR2 (ROR2);
(c) WAP four-disulfide core domain protein 2 (WFDC2);
(d) beta-2-microglobulin (beta-2-M); and/or
(e) tumor necrosis factor receptor superfamily member 1A (TNFRSF1A).

In some aspects, the therapeutic treatment is any one or more of Aimovig™ (erenumab-aooe; AMG 334), Aranesp® (darbepoetin alfa), AVSOLA™ (infliximab-axxq), BLINCYTO® (blinatumomab), Corlanor® (ivabradine), Enbrel® (etanercept), EPOGEN® (epoetin alfa), EVENITY™ (romosozumab-aqqg), IMLYGIC® (talimogene laherparepvec), Kanjinti™ (trastuzumab-anns), Kyprolis® (carfilzomib), MVASI™ (bevacizumab-awwb), Neulasta® (pegfilgrastim), NEUPOGEN® (filgrastim), Nplate® (romiplostim), Otezla® (apremilast), Parsabiv™ (etelcalcetide), Prolia® (denosumab), Repatha® (evolocumab), Sensipar® (cinacalcet), Vectibix® (panitumumab), or XGEVA® (denosumab).

In some aspects, the therapeutic treatment is a biological or a pharmaceutical drug that
(a) reduces the risk of mortality from a neoplastic disease or disorder;
(b) reduces the risk of mortality from a nervous disease or disorder;
(c) reduces the risk of mortality from a circulatory disease or disorder;
(d) reduces the risk of mortality from a respiratory disease or disorder;
(e) reduces the risk of mortality from inflammation or an inflammatory disease or disorder; and/or
(d) reduces the risk of mortality from an autoimmune disease or disorder.

In some aspects, the biological or pharmaceutical drug reduces the risk of mortality from a neoplastic disease or disorder.

In some aspects, the biological or pharmaceutical drug reduces the risk of mortality from a nervous disease or disorder.

In some aspects, the biological or pharmaceutical drug reduces the risk of mortality from a circulatory disease or disorder.

In some aspects, the biological or pharmaceutical drug reduces the risk of mortality from a respiratory disease or disorder.

In some aspects, the biological or pharmaceutical drug reduces the risk of mortality from an inflammatory disease or disorder.

In some aspects, the biological or pharmaceutical drug reduces the risk of mortality from an autoimmune disease or disorder.

In some aspects, the circulatory disease or disorder is hypercholesterolemia, myocardial infarction, and/or stroke.

In some aspects, the biological or pharmaceutical drug is evolocumab, atorvastatin, fluvastatin, lovastatin, pitavastatin, pravastatin, rosuvastatin calcium, or simvastatin.

In some aspects, the biological or pharmaceutical drug is evolocumab.

In some aspects, the respiratory disease or disorder is asthma, allergy, carbon monoxide poisoning, smoke inhalation, chronic bronchitis, emphysema, asbestos poisoning, bronchitis, pulmonary fibrosis, cystic fibrosis/bronchiectasis, lung cancer, embolism, Chronic Obstructive Pulmonary Disease (COPD), adult respiratory distress syndrome, pulmonary hypertension, Celiac's disease, pneumonitis, pneumonia, or pleural effusion.

In some aspects, the respiratory disease or disorder is asthma.

In some aspects, the biological or pharmaceutical drug is tezepelumab, omalizumab, benzalizumab, mepolizumab, or reslizumab.

In some aspects, the biological or pharmaceutical drug is tezepelumab.

In some aspects, the inflammatory disease or disorder is rheumatoid arthritis, psoriatic arthritis, plaque psoriasis, or ankylosing spondylitis.

In some aspects, the biological or pharmaceutical drug is etanercept, adalimumab, dupilumab, ustekinumab, infliximab, golimumab, or certolizumab pegol.

In some aspects, the biological or pharmaceutical drug is etanercept.

In some aspects, the reference level is a level of protein found within the range for persons identified to not be at an increased risk of mortality.

In some aspects, the methods of the disclosure comprise measuring a level of at least two, at least three, at least four, at least five, at least six, at least seven, at least eight, at least nine, at least 10, at least 11, at least 12, at least 13, at least 14, at least 15, at least 16, at least 17, at least 18, at least 19, or at least 20 proteins in the biological sample from the subject.

In some aspects, the biological sample is a blood sample.

In some aspects, the blood sample is plasma.

The disclosure provides a method for predicting increased risk of mortality of a subject, the method comprising:
measuring the level of a protein in a biological sample from the subject and comparing the level to a reference level, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSF1A);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2),
wherein the subject is determined to have an increased risk of mortality when
(i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level; and/or
(ii) the level of ANTRX2, HPGDS, ADAM22, GHR, or CILP2 is decreased relative to the reference level.

In some aspects, the methods comprise measuring the level of each of any two or more of
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; and/or
(at) WNT1-inducible-signaling pathway protein 2 (WISP-2); and
comparing the measured level of each of the proteins to the reference level for each of the proteins, wherein the subject is determined to have an increased risk of mortality when
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level; and/or
   (ii) the level of ANTRX2, HPGDS, ADAM22, GHR, or CILP2 is decreased relative to the reference level.

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) anthrax toxin receptor 2 (ANTRX2).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-2 (SPON2);
(h) serum amyloid A-1 protein (SAA1);
(i) serum amyloid A-2 protein (SAA2); and/or
(j) C5a anaphylatoxin (C5aAT).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSFiA);
(h) angiopoietin-2 (ANG2);
(i) macrophage metalloelastase (MMP12); and/or
(j) spondin-2 (SPON2).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSFiA);
(h) angiopoietin-2 (ANG2);
(i) macrophage metalloelastase (MMP12); and/or
(j) transgelin (TAGLN).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) angiopoietin-2 (ANG2);
(g) tumor necrosis factor receptor superfamily member 1A (TNFRSF1A);
(h) macrophage metalloelastase (MMP12);
(i) sushi, von Willebrand factor type A, EGF and pentraxin domain-containing protein 1 (SVEP1); or
(j) C5a anaphylatoxin (C5aAT).

In some aspects, the protein and/or proteins is
(a) growth/differentiation factor 15 (GDF15);
(b) thrombospondin-2;
(c) retinoblastoma-like protein 2;
(d) WAP four-disulfide core domain protein 2;
(e) anthrax toxin receptor 2;
(f) alpha-1-antichymotrypsin complex;
(g) spondin-2;
(h) serum amyloid A-1;
(i) serum amyloid A-2;
(j) C5a anaphylatoxin;
(k) tumor necrosis factor receptor superfamily member 1A;
(l) angiopoietin-2;
(m) macrophage metalloelastase;
(n) transgelin; and/or
(o) sushi, von Willebrand factor type A, EGF and pentraxin domain-containing protein 1.

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) alpha-1-antichymotrypsin complex (ACT COMPLEX or SERPINA3).

In some aspects, the protein and/or proteins is
(a) hematopoietic prostaglandin D synthase (HPGDS);
(b) anthrax toxin receptor 2 (ANTRX2);
(c) disintegrin and metalloproteinase domain-containing protein 22 (ADAM22); (d) growth hormone receptor (GHR); and/or
(e) insulin-like growth factor-binding protein 2 (IGFPB2).

In some aspects, the protein and/or proteins is
(a) growth/differentiation factor 15 (GDF15);
(b) WAP four-disulfide core domain protein 2 (WFDC2);
(c) cardiac troponin T (cTnT);
(d) cartilage intermediate layer protein 2 (CILP2); and/or
(e) hematopoietic prostaglandin D synthase (HPGDS).

In some aspects, the protein and/or proteins is
(a) growth/differentiation factor 15 (GDF15);
(b) WAP four-disulfide core domain protein 2 (WFDC2);
(c)N-terminal pro-BNP (NTproBNP);
(d) transmembrane emp24 domain-containing protein 10 (TMED10); and/or
(e) pancreatic ribonuclease (RNase1).

In some aspects, the protein and/or proteins is
(a) growth/differentiation factor 15 (GDF15);
(b) tyrosine-protein kinase transmembrane receptor ROR2 (ROR2);
(c) WAP four-disulfide core domain protein 2 (WFDC2);
(d) beta-2-microglobulin (beta-2-M); and/or
(e) tumor necrosis factor receptor superfamily member 1A (TNFRSF1A).

In some aspects, the subject has an increased risk of mortality within 2 years.

In some aspects, the subject has an increased risk of mortality within 5 years.

In some aspects, the subject has an increased risk of mortality within 10 years.

In some aspects, the subject has an increased risk of mortality within 15 years.

In some aspects, the subject has an increased risk of mortality from a neoplastic disease or disorder.

In some aspects, the subject has an increased risk of mortality from a nervous system disease or disorder.

In some aspects, the subject has an increased risk of mortality from a circulatory system disease or disorder.

In some aspects, the subject has an increased risk of mortality from a respiratory system disease or disorder.

In some aspects, the reference level is a mean level of the biomarker in a biological sample from a population of subjects determined to be healthy or not to be at an increased risk of mortality.

In some aspects, the methods of the disclosure comprise measuring the level of at least two, at least three, at least four, at least five, at least six, at least seven, at least eight, at least nine, at least 10, at least 11, at least 12, at least 13, at least 14, at least 15, at least 16, at least 17, at least 18, at least 19, or at least 20 proteins in the biological sample from the subject.

In some aspects, the biological sample is a blood sample.

In some aspects, the blood sample is plasma.

In some aspects, the mortality is all-cause mortality.

In some aspects, wherein when the subject is predicted to have an increased risk of mortality, the methods of the disclosure further comprise administering a therapeutic treatment to the subject to reduce the increased risk.

In some aspects, the therapeutic treatment is a dietary restriction, a fitness regimen, or the supplementation of diet.

In some aspects, the therapeutic treatment is any one or more of Aimovig™ (erenumab-aooe; AMG 334), Aranesp® (darbepoetin alfa), AVSOLA™ (infliximab-axxq), BLINCYTO® (blinatumomab), Corlanor® (ivabradine), Enbrel® (etanercept), EPOGEN®(epoetin alfa), EVENITY™ (romosozumab-aqqg), IMLYGIC® (talimogene laherparepvec), Kanjinti™ (trastuzumab-anns), Kyprolis® (carfilzomib), MVASI™ (bevacizumab-awwb), Neulasta® (pegfilgrastim), NEUPOGEN® (filgrastim), Nplate® (romiplostim), Otezla® (apremilast), Parsabiv™ (etelcalcetide), Prolia® (denosumab), Repatha® (evolocumab), Sensipar® (cinacalcet), Vectibix® (panitumumab), or XGEVA® (denosumab).

In some aspects, the therapeutic treatment is a biological or a pharmaceutical drug that
(a) reduces the risk of mortality from a neoplastic disease or disorder;
(b) reduces the risk of mortality from a nervous disease or disorder;
(c) reduces the risk of mortality from a circulatory disease or disorder;
(d) reduces the risk of mortality from a respiratory disease or disorder;
(e) reduces the risk of mortality from inflammation or an inflammatory disease or disorder; and/or
(f) reduces the risk of mortality from an autoimmune disease or disorder.

In some aspects, the biological or pharmaceutical drug reduces the risk of mortality from a neoplastic disease or disorder.

In some aspects, the biological or pharmaceutical drug reduces the risk of mortality from a nervous disease or disorder.

In some aspects, the biological or pharmaceutical drug reduces the risk of mortality from a circulatory disease or disorder.

In some aspects, the biological or pharmaceutical drug reduces the risk of mortality from a respiratory disease or disorder.

In some aspects, the biological or pharmaceutical drug reduces the risk of mortality from an inflammatory disease or disorder.

In some aspects, the biological or pharmaceutical drug reduces the risk of mortality from an autoimmune disease or disorder.

In some aspects, the circulatory disease or disorder is hypercholesterolemia, myocardial infarction, and/or stroke.

In some aspects, the biological or pharmaceutical drug is evolocumab, atorvastatin, fluvastatin, lovastatin, pitavastatin, pravastatin, rosuvastatin calcium, or simvastatin.

In some aspects, the biological or pharmaceutical drug is evolocumab.

In some aspects, the respiratory disease or disorder is asthma, allergy, carbon monoxide poisoning, smoke inhalation, chronic bronchitis, emphysema, asbestos poisoning, bronchitis, pulmonary fibrosis, cystic fibrosis/bronchiectasis, lung cancer, embolism, Chronic Obstructive Pulmonary Disease (COPD), adult respiratory distress syndrome, pulmonary hypertension, Celiac's disease, pneumonitis, pneumonia, or pleural effusion.

In some aspects, the respiratory disease or disorder is asthma.

In some aspects, the biological or pharmaceutical drug is tezepelumab, omalizumab, benzalizumab, mepolizumab, or reslizumab.

In some aspects, the biological or pharmaceutical drug is tezepelumab.

In some aspects, the inflammatory disease or disorder is rheumatoid arthritis, psoriatic arthritis, plaque psoriasis, or ankylosing spondylitis.

In some aspects, the biological or pharmaceutical drug is etanercept, adalimumab, dupilumab, ustekinumab, infliximab, golimumab, or certolizumab pegol.

In some aspects, the biological or pharmaceutical drug is etanercept.

In some aspects, the subject is a human subject.

The disclosure provides a kit comprising reagents for measuring a level a protein in a biological sample from a subject, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSF1A);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2).

In some aspects, the kit comprising reagents and/or a means for measuring the levels of two or more proteins in a biological sample from a subject, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2).

In some aspects, the kit further comprises reagents and/or a means for comparing the measured level of the protein or proteins in the biological sample with a reference level of the protein or proteins.

In some aspects, the kit further comprises instructions for use. In some aspects, the instructions for use include an instruction protocol for measuring the level of the protein and/or proteins and/or comparing the level of the protein and/or proteins to a reference level for each protein.

The disclosure provides uses of a biomarker or a combination of biomarkers, e.g., proteins or nucleic acids, alone and in combination, as predictors of increased risk of mortality and/or increased risk of poor or declining health. In some aspects, the uses are for predicting an increased or decreased risk of mortality, and/or for predicting an improvement or decline health of a subject. The disclosure also provides uses of a therapeutic treatment to a subject identified as having an increased risk of mortality and/or an increased risk of poor or declining health. In some aspects, a biomarker or a combination of biomarkers is used to determine the effect of a therapeutic treatment on the subject's risk of mortality and/or poor health.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the disclosure will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The following Detailed Description, given by way of example, but not intended to limit the disclosure or methods of the disclosure to specific embodiments described, may be understood in conjunction with the accompanying Figures, incorporated herein by reference, in which:

FIG. 1A-D depicts the discriminatory power of prediction models, by using a receiver operating characteristics (ROC) curve and the area under the curve (AUC) for participants of all ages (FIG. 1A-B) and restricted to those 60 years or older (FIG. 1C-D).

FIG. 2A-D shows the curves plotted separately for the four prediction models. By splitting the Kaplan-Meier curves by quantiles of predicted ten-year risk, the proteins' discriminative power becomes evident.

FIG. 3 shows the predicted five-year risk of all-cause mortality split by survival status after five years from plasma collection. Participants who died within five years are also shown separately for five cause-of-death categories; neoplasms, nervous system, circulatory system, respiratory system, and other. All five cause-of-death categories showed a higher predicted risk for the protein model than the baseline model, although the difference varied between categories.

FIG. 4 shows CV 5-year mortality in view of age, sex, baseline, polygenic risk score (PRS), triglyceride level (Tri), creatinine level (Cre), platelet numbers (Pla) and combinations of these factors.

FIG. 5A-B shows CV 5-year mortality using various models. Logistic regression with an L1 penalty was used for the final model since it used the fewest features (FIG. 5A-B). For the age and sex model, baseline model, and age, sex, and one protein model, logistic regression was used. L2 penalty was added when prediction with age, sex, and 1-100 preselected proteins was performed.

FIG. 6A-D shows that a visual examination showed all models reasonably well-calibrated, allowing predicted risk values to be interpreted directly as probabilities.

FIG. 7 shows that the proteins with the highest associations were rather correlated. For the top ten proteins associating with all-cause mortality within five years, most intercorrelations after correcting for age and sex were 0.4-0.6. In contrast, the average correlation between any protein pair of the 4,905 proteins was 0.34.

FIG. 8A-B shows that most of the discrimination performance can be achieved by using only a few proteins in addition to and age and sex. Five, ten, or twenty proteins, selected with a forward selection, yielded AUCs of 0.905, 0.910, and 0.912 for five-year prediction (FIG. 8A) and 0.913, 0.917, and 0.919 for ten-year prediction (FIG. 8B).

### DETAILED DESCRIPTION

The disclosure relates to the identification of various biomarkers, e.g., proteins or nucleic acids, alone and in combination, as predictors of increased mortality risk and/or increased risk of poor or declining health. More specifically, the disclosure provides fast and robust methods of predicting an increased or decreased risk of mortality, or an improvement or decline in mortality risk and/or health, by measuring a level of at least one biomarker, e.g., a level of protein or nucleic acid, in a biological sample from a subject, wherein a change in the level of the biomarker of the subject compared to a reference level, or compared to a level of the biomarker before and after treatment with a therapeutic agent, regimen, or event indicates whether the subject is exhibiting an improvement or decline in health, or an increased or decreased risk of mortality. The disclosure provides methods for decreasing mortality risk and/or improving health in a subject. The disclosure also provides methods for treating a subject having an increased risk of mortality and/or poor health and then determining the effect of a therapeutic treatment on the subject's risk of mortality and/or poor health. Thus, in some aspects, the disclosure provides methods of using one or more biomarkers to determine the risk of mortality, detect an increase or decrease over time in the risk of mortality, or detect an improvement or decline in health of a subject that occurs, for example, over time, in response to therapeutic intervention, or as a result of changes in diet, fitness, or other lifestyle changes. In some aspects, the therapeutic treatment is a diet restriction, a fitness regimen, and/or a supplementation of diet. In some other aspects, the disclosure provides methods for treating a subject to reduce the mortality risk of the subject. Additionally, the disclosure provides methods for using changes in the risk of mortality of a subject to monitor the effectiveness of a therapeutic treatment, dietary restrictions, fitness regimen, or other intervention in a subject, and for continuing, discontinuing, or altering the treatment, restrictions, regimen, or intervention accordingly. Further, the disclosure provides methods for predicting effectiveness of a therapeutic treatment in a subject determined to be at an increased risk of mortality and subsequently treating the subject with the therapeutic treatment if the biomarker level is indicative of effectiveness of the treatment of the subject.

Before any embodiments of the subject matter of the disclosure are explained in detail, it is to be understood that the disclosure is not limited in its application to the details of construction and the arrangement of components set forth in the following description or illustrated in the figures and examples. Accordingly, the disclosure embraces other embodiments and is practiced or carried out in various ways.

The section headings as used herein are for organizational purposes only and are not to be construed as limiting the subject matter described.

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

The terms “protein,” “polypeptide,” and “peptide” are used interchangeably herein to refer to a polymer of amino acid residues linked via peptide bonds. The term “protein” typically refers to large polypeptides. The term “peptide” typically refers to short polypeptides. The term “protein”, as used herein, includes a fragment of a protein or any portion of the protein smaller than the full-length protein or protein expression product. Fragments are deletion analogs of the full-length protein wherein one or more amino acid residues have been removed from the amino terminus (protein) and/or the carboxy terminus of the full-length protein.

The term “nucleic acid” or “nucleic acid sequence” or “nucleic acid molecule” refers to deoxyribonucleotides or ribonucleotides and polymers thereof in either single- or double-stranded form. The term nucleic acid is used interchangeably with gene, deoxyribonucleic acid, complementary DNA (cDNA), ribonucleic acid, messenger RNA (mRNA), oligonucleotide, and polynucleotide. The term “nucleic acid”, as used herein, includes a fragment of a nucleic acid or any portion of the nucleic acid smaller than the full-length nucleic acid. Fragments include deletion analogs of the full-length nucleic acid wherein one or more nucleotides have been removed from the 5′ end and/or the 3′ end of the full-length nucleic acid.

A “biomarker” in the context of the disclosure encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. In some aspects, therefore, a biomarker includes a protein or a fragment thereof or a nucleic acid or a fragment thereof. In additional aspects, one or more biomarkers are measured together to provide an array for the prediction that the subject will positively respond to a treatment regimen. In exemplary aspects of the disclosure, a biomarker is a protein.

A biomarker, e.g., protein or nucleic acid, of the disclosure is one or more of the following:
Growth/differentiation factor 15 (GDF15),
Thrombospondin-2 (TSP-2 or THBS2),
Retinoblastoma-like protein 2 (RBP2),
WAP four-disulfide core domain protein 2 (WFDC2),
Anthrax toxin receptor 2 (ANTRX2),
Alpha-1-antichymotrypsin complex (ACT COMPLEX or SERPINA3),
Spondin-1 (SPON1),
Spondin-2 (SPON2),
Serum amyloid A-1 protein (SAA1),
Serum amyloid A-2 protein (SAA2),
C5a anaphylatoxin (C5aAT),
Tumor necrosis factor receptor superfamily member 1A (TNFRSF1A),
Angiopoietin-2 (ANG2 or ANGPT2),
Macrophage metalloelastase (MMP12),
Transgelin (TAGLN),
Sushi, von Willebrand factor type A, EGF and pentraxin domain-containing protein 1 (SVEP1),
Hematopoietic prostaglandin D synthase (HPGDS),
Disintegrin and metalloproteinase domain-containing protein 22 (ADAM22),
Growth hormone receptor (GHR),
Insulin-like growth factor-binding protein 2 (IGFPB2),
N-terminal pro-BNP (NTproBNP),
Transmembrane emp24 domain-containing protein 10 (TMED10),
Pancreatic ribonuclease (or ribonuclease pancreatic or ribonuclease A (RNase A) or ribonuclease 1 (RNase1)),
Troponin T. cardiac muscle (or cardiac troponin T (cTnT)),
Cartilage intermediate layer protein 2 (CILP2),
Tyrosine-protein kinase transmembrane receptor ROR2 (or neurotrophic tyrosine kinase or receptor-related 2 or ROR2),
Beta-2-microglobulin (beta-2-M),
Insulin-like growth factor-binding protein 6 (IGFPB6),
Tetranectin (TN),
dCTP pyrophosphatase 1 (DCTPP1),
Tumor necrosis factor receptor superfamily member EDAR (Ectodysplasin (EDA) receptor or EDAR),
Erythropoietin (EPO),
Complement component C7 (C7),
L-Selectin (CD62L),
Netrin receptor UNC5B (UNCB5),
Brorin,
Epidermal growth factor receptor (EGFR or ERBB or ERBB1 or HER1 or NISBD2 or PIG61 or mENA),
Serine protease inhibitor Kazal-type 5 (SPINK5),
Immunoglobulin superfamily member 3 (IGSF3),
Fc receptor-like protein 1 (FCRL1),
Pleiotrophin (PTN),
RGM domain family member B (RGMB),
Ephrin type-B receptor 2 (EPHB2),
Triggering receptor expressed on myeloid cells 1 (TREM-1),
Elafin (or skin-derived anti-leukoprotease (SKALP) or elastase-specific inhibitor (ESI)), and/or
WNT1-inducible-signaling pathway protein 2 (WISP-2).

In various embodiments, therefore, a protein of the disclosure is any one or more of the following:
Growth/differentiation factor 15 (GDF15),
Thrombospondin-2 (TSP-2 or THBS2),
Retinoblastoma-like protein 2 (RBP2),
WAP four-disulfide core domain protein 2 (WFDC2),
Anthrax toxin receptor 2 (ANTRX2),
Alpha-1-antichymotrypsin complex (ACT COMPLEX or SERPINA3),
Spondin-1 (SPON1),
Spondin-2 (SPON2),
Serum amyloid A-1 protein (SAA1),
Serum amyloid A-2 protein (SAA2),
C5a anaphylatoxin (C5aAT),
Tumor necrosis factor receptor superfamily member 1A (TNFRSF1A),
Angiopoietin-2 (ANG2 or ANGPT2),
Macrophage metalloelastase (MMP12),
Transgelin (TAGLN),
Sushi, von Willebrand factor type A, EGF and pentraxin domain-containing protein 1 (SVEP1),
Hematopoietic prostaglandin D synthase (HPGDS),
Disintegrin and metalloproteinase domain-containing protein 22 (ADAM22),
Growth hormone receptor (GHR),
Insulin-like growth factor-binding protein 2 (IGFPB2),
N-terminal pro-BNP (NTproBNP),
Transmembrane emp24 domain-containing protein 10 (TMED10),
Pancreatic ribonuclease (or ribonuclease pancreatic or ribonuclease A (RNase A) or ribonuclease 1 (RNase1)),
Troponin T. cardiac muscle (or cardiac troponin T (cTnT)),
Cartilage intermediate layer protein 2 (CILP2),
Tyrosine-protein kinase transmembrane receptor ROR2 (or neurotrophic tyrosine kinase or receptor-related 2 or ROR2),
Beta-2-microglobulin (beta-2-M),
Insulin-like growth factor-binding protein 6 (IGFPB6),
Tetranectin (TN),
dCTP pyrophosphatase 1 (DCTPP1),
Tumor necrosis factor receptor superfamily member EDAR (Ectodysplasin (EDA) receptor or EDAR),
Erythropoietin (EPO),
Complement component C7 (C7),
L-Selectin (CD62L),
Netrin receptor UNC5B (UNCB5),
Brorin,
Epidermal growth factor receptor (EGFR or ERBB or ERBB1 or HER1 or NISBD2 or PIG61 or mENA),
Serine protease inhibitor Kazal-type 5 (SPINK5),
Immunoglobulin superfamily member 3 (IGSF3),
Fc receptor-like protein 1 (FCRL1),
Pleiotrophin (PTN),
RGM domain family member B (RGMB),
Ephrin type-B receptor 2 (EPHB2),
Triggering receptor expressed on myeloid cells 1 (TREM-1),
Elafin (or skin-derived anti-leukoprotease (SKALP) or elastase-specific inhibitor (ESI)), and/or
WNT1-inducible-signaling pathway protein 2 (WISP-2).

In some aspects, the methods, kits, and uses of the disclosure comprise a biomarker. In some aspects, the methods, kits, and uses comprise at least two, at least three, at least four, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, and 46 biomarkers of the disclosure.

In exemplary aspects, the term “GDF15”, as used herein, refers to a GDF15 protein or nucleic acid; the term “TSP-2”, as used herein, refers to a TSP-2 protein or nucleic acid; the term “RBP2”, as used herein, refers to an RBP2 protein or nucleic acid; the term “WFDC2”, as used herein, refers to a WFDC2 protein or nucleic acid; the term “ANTRX2”, as used herein, refers to an ANTRX2 protein or nucleic acid; the term “ACT COMPLEX” or “SERPINA3”, as used herein, refers to an ACT COMPLEX or SERPINA3 protein or nucleic acid; the term “SPON1”, as used herein, refers to a SPON1 protein or nucleic acid; the term “SPON2”, as used herein, refers to a SPON2 protein or nucleic acid; the term “SAA1”, as used herein, refers to an SAA1 protein or nucleic acid; the term “SAA2”, as used herein, refers to an SAA2 protein or nucleic acid; the term “C5aAT”, as used herein, refers to a C5aAT protein or nucleic acid; the term “TNFRSF1A”, as used herein, refers to a TNFRSF1A protein or nucleic acid; the term “ANG2”, as used herein, refers to an ANG2 protein or nucleic acid; the term “MMP12”, as used herein, refers to an MMP12 protein or nucleic acid; the term “TAGLN”, as used herein, refers to a TAGLN protein or nucleic acid; the term “SVEP1”, as used herein, refers to a SVEP1 protein or nucleic acid; the term “HPGDS”, as used herein, refers to a HPGDS protein or nucleic acid; the term “ADAM22”, as used herein, refers to an ADAM22 protein or nucleic acid; the term “GHR”, as used herein, refers to a GHR protein or nucleic acid; the term “IGFPB2”, as used herein, refers to an IGFPB2 protein or nucleic acid; the term “NTproBNP”, as used herein, refers to an NTproBNP protein or nucleic acid; the term “TMED10”, as used herein, refers to a TMED10 protein or nucleic acid; the term “RNase1”, as used herein, refers to an RNase1 protein or nucleic acid; the term “cTnT”, as used herein, refers to a cTnT protein or nucleic acid; the term “CILP2”, as used herein, refers to a CILP2 protein or nucleic acid; the term “ROR2”, as used herein, refers to a ROR2 protein or nucleic acid; the term “beta-2-M”, as used herein, refers to a beta-2-M protein or nucleic acid; the term “IGFPB6”, as used herein, refers to an IGFPB6 protein or nucleic acid; the term “TN”, as used herein, refers to a TN protein or nucleic acid; the term “DCTPP1”, as used herein, refers to a DCTPP1 protein or nucleic acid; the term “EDAR”, as used herein, refers to an EDAR protein or nucleic acid; the term “EPO”, as used herein, refers to an EPO protein or nucleic acid; the term “C7”, as used herein, refers to a C7 protein or nucleic acid; the term “CD62L”, as used herein, refers to a CD62L protein or nucleic acid; the term “UNCB5”, as used herein, refers to a UNCB5 protein or nucleic acid; the term “brorin”, as used herein, refers to a brorin protein or nucleic acid; the term “EGFR”, as used herein, refers to an EGFR protein or nucleic acid; the term “SPINK5”, as used herein, refers to a SPINK5 protein or nucleic acid; the term “IGSF3”, as used herein, refers to an IGSF3 protein or nucleic acid; the term “FCRL1”, as used herein, refers to an FCRL1 protein or nucleic acid; the term “PTN”, as used herein, refers to a PTN protein or nucleic acid; the term “RGMB”, as used herein, refers to an RGMB protein or nucleic acid; the term “EPHB2”, as used herein, refers to an EPHB2 protein or nucleic acid; the term “TREM-1”, as used herein, refers to a TREM-1 protein or nucleic acid; the term “elafin”, as used herein, refers to an elafin protein or nucleic acid; and the term “WISP-2”, as used herein, refers to a WISP-2 protein or nucleic acid.

The disclosure includes the use of any one biomarker or combination of biomarkers described herein in any of the disclosed methods, kits, uses and the like. For example, the disclosure, in various aspects, includes any biomarker or combination of the 46 biomarkers of the disclosure, either each on its own and/or in any combination of at least one, at least two, at least three, at least four, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, and 46.

Because a table displaying all possible combinations of the biomarkers of the disclosure would require over 40-50 pages of text, Table 1 is provided below showing, for illustrative purposes only, the theoretical combination of five biomarkers. The disclosure contemplates a similar means and methodology for combining all possible combinations of the 46 biomarkers of the disclosure. Thus, the disclosure includes thousands of possible combinations of biomarkers as theoretically illustrated.

TABLE 1
Biomarkers and Combinations of Biomarkers.

| | Combination of Biomarkers for Illustrative Purposes Only | | | | | |
|---|---|---|---|---|---|---|
| Row No. | 1 | 2 | 3 | 4 | 5 | . . . |

| | | | | | |
|---|---|---|---|---|---|
| 1 | X | | | | |
| 2 | X | X | | | |
| 3 | X | | X | | |
| 4 | X | | | X | |
| 5 | X | | | | X |
| 6 | | X | | | |
| 7 | | X | X | | |
| 8 | | X | | X | |
| 9 | | X | | | X |
| 10 | | | X | | |
| 11 | | | X | X | |
| 12 | | | X | | X |
| 13 | | | | X | |
| 14 | | | | X | X |
| 15 | | | | | X |
| 16 | X | X | X | | |
| 17 | X | X | | X | |
| 18 | X | X | | | X |
| 19 | | X | X | X | |
| 20 | | | X | X | X |
| 21 | | X | X | | X |
| 22 | X | X | X | X | |
| 23 | X | X | | X | X |
| 24 | X | | X | X | X |
| 25 | | X | X | X | X |
| 26 | X | X | X | X | X |
| . . . | | | | | |

In the disclosure, an increase or decrease in the level of each of the 46 biomarkers is each independently correlated with an increased or decreased risk of mortality or an increased or decreased risk of poor or declining health. For each biomarker (protein or gene), the expression level was correlated with an increasing or decreasing risk of mortality at 2 years, 5 years, 10 years, or 15 years, adjusting for the body mass index (BMI), gender, and age of the patient. Each of these 46 biomarkers was selected because they were among the top 46 most significant proteins associated with an increased or decreased risk of mortality.

For example, Table 5 shows the most significant proteins associated with an increased risk of mortality at 2, 5, 10 or 15 years. In an exemplary aspect, Table 5 shows that having a greater level of GDF15 protein provides a subject with an increased risk of mortality; and having a greater level of anthrax receptor 2 provides a subject with a decreased risk of mortality. For proteins that are not predictive or are less predictive of mortality, their beta value is 0 or closer to 0. Table 5, in one aspect, shows that GDF15 is a protein that has value as a predictor of death within 2, 5, 10, or 15 years. The 1.35 beta value of GDF for death within 2 years corresponds to an odds ratio of about 3.9. That is, for every standard deviation that GDF goes up by, a subject is 3.9 times more likely to die within 2 years.

In various aspects of the disclosure, a beta value is measured. Beta (p) (alternately known as “beta value” or “beta coefficient”) refers to the probability of Type II error in a statistical hypothesis test. Frequently, the power of a test, equal to 1-3 rather than p itself, is referred to as a measure of quality for a hypothesis test. The beta value is directly related to the power of a test. Power relates to how likely a test is to distinguish an actual effect from one you could expect to happen by chance alone. The beta value can be negative or positive, and have a t-value and significance of the t-value associated with each. The beta coefficient is the degree of change in the outcome variable for every 1-unit of change in the predictor variable. The t-test assesses whether the beta coefficient is significantly different from zero. If the beta value is not statistically significant (i.e., the t-value is not significant), the variable does not significantly predict the outcome. If the beta value is significant, examine the sign of the beta. If the beta coefficient is positive, the interpretation is that for every 1-unit increase in the predictor variable, the outcome variable will increase by the beta coefficient value. If the beta coefficient is negative, the interpretation is that for every 1-unit increase in the predictor variable, the outcome variable will decrease by the beta coefficient value.

In some aspects of the disclosure, the level of a biomarker or the combination of biomarkers is measured in a sample from a subject and compared to a reference level. A “reference level”, as used herein, provides a known comparator for measuring the level or amount of biomarker, e.g., protein or nucleic acid, present in a biological sample from a subject.

In some aspects of the disclosure, the reference level is a mean level of the biomarker in a biological sample from a population of subjects determined to be healthy or not to be at an increased risk of mortality. In some aspects, the population of subjects is, optionally, matched to the subject in other parameters, such as one or more of the following: age, sex, body mass index (BMI) and the like. In various aspects, the level of the biomarker is a relative level. In some aspects, the level of the biomarker is an absolute level. In some aspects, the level of the biomarker is within the range of the mean level of the biomarker in a population of healthy subjects or subjects determined not to be at risk of mortality.

In some aspects of the disclosure, the level of a biomarker or the levels of a combination of biomarkers is measured in a sample from a subject before and after a therapeutic treatment. Thus, a first level and a second level is measured. In such aspects, the first level is the level of the biomarker or the levels of each of the biomarkers in the sample from the subject before the therapeutic treatment. In such aspects, the second level is the level of the biomarker or the levels of each of the biomarkers in the sample from the subject after the therapeutic treatment. In some aspects, the level of a biomarker or the levels of multiple biomarkers is measured at multiple times throughout the course of a therapeutic treatment. In such aspects, the reference level is the level of the biomarker in the subject before the therapeutic treatment or agent is administered (i.e., baseline).

In some aspects, the population of subjects is at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, at least about 600, at least about 700, at least about 800, at least about 900, at least about 1000 subjects, at least about 2000 subjects, at least about 3000 subjects, at least about 4000 subjects, at least about 5000 subjects, at least about 6000 subjects, at least about 7000 subjects, at least about 8000 subjects, at least about 9000 subjects, at least about 10000 subjects, at least about 15000 subjects, at least about 20000 subjects, or at least about 25000 subjects.

In various aspects, an increased level of biomarker is a level greater than the reference level. In various aspects, an increase in the level of the biomarker in a subject is at least or about 1% greater, at least or about 2% greater, at least or about 3% greater, at least or about 4% greater, at least or about 5% greater, at least or about 6% greater, at least or about 7% greater, at least or about 8% greater, at least or about 9% greater, at least or about 10% greater, at least or about 11% greater, at least or about 12% greater, at least or about 13% greater, at least or about 14% greater, at least or about 15% greater, at least or about 16% greater, at least or about 17% greater, at least or about 18% greater, at least or about 19% greater, at least or about 20% greater, at least or about 21% greater, at least or about 22% greater, at least or about 23% greater, at least or about 24% greater, at least or about 25% greater, at least or about 26% greater, at least or about 27% greater, at least or about 28% greater, at least or about 29% greater, at least or about 30% greater, at least or about 35% greater, at least or about 40% greater, at least or about 45% greater, at least or about 50% greater, at least or about 55% greater, at least or about 60% greater, at least or about 65% greater, at least or about 70% greater, at least or about 75% greater, at least or about 80% greater, at least or about 85% greater, at least or about 90% greater, at least or about 95% greater, at least or about 100% greater, at least or about greater than 100% greater than the reference level, or the level of the biomarker before treatment.

In additional aspects, an increase in the level of the biomarker in a subject is at least or about 1/10 greater, at least or about 1/9 greater at least or about ⅛ greater, at least or about 1/7 greater, at least or about ⅙ greater, at least or about ⅕ greater, at least or about ¼ greater, at least or about ⅓ greater, at least or about ½ greater, at least or about 1 times greater, at least or about 1.5 times greater, at least or about 2.0 times greater, at least or about 2.5 times greater, at least or about 3.0 times greater, at least or about 3.5 times greater, at least or about 4.0 times greater, at least or about 4.5 times greater, at least or about 5 times greater, at least or about 10 times greater, at least or about 15 times greater, at least or about 20 times greater, at least or about 25 times greater, at least or about 30 times greater, at least or about 35 times greater, at least or about 40 times greater, at least or about 45 times greater, at least or about 50 times greater, at least or about 55 times greater, at least or about 60 times greater, at least or about 65 times greater, at least or about 70 times greater, at least or about 75 times greater, at least or about 80 times greater, at least or about 85 times greater, at least or about 90 times greater, at least or about 100 times greater, or at least or about greater than about 100 times than the reference level, or the level of the biomarker before treatment.

In various aspects, a decreased level of a biomarker is a level lesser than the reference level. In various aspects, a decrease in the level of the biomarker is at least or about 1% lesser, at least or about 2% lesser, at least or about 3% lesser, at least or about 4% lesser, at least or about 5% lesser, at least or about 6% lesser, at least or about 7% lesser, at least or about 8% lesser, at least or about 9% lesser, at least or about 10% lesser, at least or about 11% lesser, at least or about 12% lesser, at least or about 13% lesser, at least or about 14% lesser, at least or about 15% lesser, at least or about 16% lesser, at least or about 17% lesser, at least or about 18% lesser, at least or about 19% lesser, at least or about 20% lesser, at least or about 21% lesser, at least or about 22% lesser, at least or about 23% lesser, at least or about 24% lesser, at least or about 25% lesser, at least or about 26% lesser, at least or about 27% lesser, at least or about 28% lesser, at least or about 29% lesser, at least or about 30% lesser, at least or about 35% lesser, at least or about 40% lesser, at least or about 45% lesser, at least or about 50% lesser, at least or about 55% lesser, at least or about 60% lesser, at least or about 65% lesser, at least or about 70% lesser, at least or about 75% lesser, at least or about 80% lesser, at least or about 85% lesser, at least or about 90% lesser, at least or about 95% lesser, at least or about 100% lesser, or about lesser than 100% lesser than the reference level, or the level of the biomarker before treatment.

In additional aspects, a decrease in the level of the biomarker in a subject is at least or about 1/10 lesser, at least or about 1/9 lesser at least or about ⅛ lesser, at least or about 1/7 lesser, at least or about ⅙ lesser, at least or about ⅕ lesser, at least or about ¼ lesser, at least or about ⅓ lesser, at least or about ½ lesser, at least or about 1 times lesser, at least or about 1.5 times lesser, at least or about 2.0 times lesser, at least or about 2.5 times lesser, at least or about 3.0 times lesser, at least or about 3.5 times lesser, at least or about 4.0 times lesser, at least or about 4.5 times lesser, or at least or about 5 times lesser, at least or about 10 times lesser, at least or about 15 times lesser, at least or about 20 times lesser, at least or about 25 times lesser, at least or about 30 times lesser, at least or about 35 times lesser, at least or about 40 times lesser, at least or about 45 times lesser, at least or about 50 times lesser, at least or about 55 times lesser, at least or about 60 times lesser, at least or about 65 times lesser, at least or about 70 times lesser, at least or about 75 times lesser, at least or about 80 times lesser, at least or about 85 times lesser, at least or about 90 times lesser, at least or about 100 times lesser, or at least or about greater than about 100 times lesser than the reference level, or the level of the biomarker before treatment.

Detecting and Measuring Biomarker Level

In various aspects of the disclosure, level of the protein biomarker is detected or quantitatively measured in a biological sample by any suitable means known in the art for quantifying protein including, but not limited to, an immunoassay (e.g., ELISA, RIA), immunoturbidimetry, rapid immunodiffusion, laser nephelometry, visual agglutination, quantitative Western blot analysis, multiple reaction monitoring-mass spectrometry (MRM Proteomics), Lowry assay, Bradford assay, BCA assay, UV spectroscopic assays, such as a UV spectroscopic assay, surface plasmon resonance, and SOMAscan® assay. In various aspects, therefore, the level measured is an absolute level or is a relative level.

In exemplary aspects of the disclosure, aptamers, including SOMAmers (SomaLogic) are used to measure the relative level of protein a SOMAscan® assay. The SOMAscan® assay measures native proteins in complex matrices by transforming each individual protein concentration into a corresponding SOMAmer reagent concentration, which is then quantified by standard DNA techniques such as microarrays or qPCR (SOMAscan® Proteomic AssayTechnical White Paper (https-colon-slash-slash-somalogic.com/wp-content/uploads/2017/06/SSM-002-Technical-White-Paper_010916_LSM1.pdf)). The SOMAscan® assay quantitatively transforms the proteins present in a biological sample into a specific SOMAmer-based DNA signal. A SOMAmer-protein binding step is followed by a series of partitioning and wash steps that converts relative protein concentrations into measurable nucleic acid signals that are quantified using DNA detection technology, which for the SOMAscan assay with over 1,300 SOMAmer reagents is by hybridization to custom DNA microarrays. Assays with smaller numbers of SOMAmer reagents (i.e., 1-100, called “SOMAmer panels”), have been quantified by either qPCR or Luminex beads using sequences complementary to the SOMAmer reagent sequences. Assay details are provided in Appendix A of SOMAscan® Proteomic AssayTechnical White Paper (https-colon-slash-slash-somalogic.com/wp-content/uploads/2017/06/SSM-002-Technical-White-Paper_010916_LSM1.pdf). Whichever detection method is used, the readout in relative fluorescent units (RFU) is directly proportional to the amount of target protein in the initial sample, as informed by a standard curve generated for each protein SOMAmer pair. The use of a SOMAscan® assay to measure relative protein level and increases and decreases in protein level is known in the art (Gold et al., PLos ONE 5(12):e15004; Christiansson et al., EuPA Open Proteomics 3: 37-47, 2014; Giudice et al., Exp. Hematol. 68:38-50, 2018; Kim et al., Scientific Reports 8:8382, 2018)ln various aspects of the disclosure, level of the nucleic acid biomarker is detected or quantitatively measured in a biological sample by any suitable means known in the art for quantifying nucleic acid including, but not limited to, RNA sequencing (RNA-seq), high-throughput sequencing (HT-seq), PCR, quantitiative PCR, qT-PCR, RT-qPCR, digital PCR, real-time PCR, direct digital quantification, serial analysis of gene expression (SAGE), nucleic acid sequence-based amplification (NASBA), transcription-mediated amplification (TMA), branched DNA (bDNA) assays, and/or Northern or Southern blotting.

In various aspects, any of these methods is performed on protein or nucleic acid isolated from or present in a biological sample obtained from a human subject. In some aspects, the biological sample is blood. In some aspects, the sample is serum. In exemplary aspects, the biological sample is plasma.

In some aspects, the sample is taken prior to treatment to measure the level of the biomarker before treatment. In some aspects, the sample is taken before and after treatment with a therapeutic agent to measure the level of the biomarker before or after treatment. In some aspects, the sample is taken over multiple time points to monitor the effect of a therapeutic treatment over time. Thus, in some aspects, a first value of the biomarker, e.g., protein level, is compared to a second value or is compared to multiple values taken over a period of time. In some aspects, the sample is taken before, during, and after treatment with a therapeutic agent to measure the level of the biomarker before, during, or after treatment to determine the effect of a biomarker during treatment. In some aspects, patients have a full clinical assessment at baseline (i.e., time 0) or prior to a first treatment with a therapeutic agent.

“Measuring” or “measurement” means assessing the presence, quantity or level of a biomarker, e.g. a protein, within a clinical or subject-derived sample, including the derivation of qualitative or quantitative concentration levels of such substance, or otherwise evaluating the values or categorization of a subject's clinical parameters.

Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

The terms “level” and “amount” are used herein interchangeably to mean the concentration of biomarker, e.g., protein or nucleic acid, present in a biological sample. In some aspects of the disclosure, the biological sample is a blood sample. In some aspects, the blood sample is plasma or serum. In some aspects, protein and/or nucleic acid is prepared from the sample and the “level” and/or “amount” is the level or amount of a particular protein and/or nucleic acid of interest. In some aspects, it is the level or amount of the protein or nucleic acid biomarker.

In some aspects, the methods include measuring the level of at least one or more of GDF15, TSP-2, RBP2, WFDC2, ANTRX2, ACT COMPLEX or SERPINA3, SPON1, SPON2, SAA1, SAA2, C5aAT, TNFRSF1A, ANG2, MMP12, TAGLN, SVEP1, HPGDS, ADAM22, GHR, IGFPB2, NTproBNP, TMED10, RNase1, cTnT, CILP2, ROR2, beta-2-M, IGFPB6, TN, DCTPP1, EDAR, EPO, C7, CD62L, UNCB5, brorin, EGFR, SPINK5, IGSF3, FCRL1, PTN, RGMB, EPHB2, TREM-1, elafin, and/or WISP-2. In some aspects, the level is a protein level in a biological sample. In some aspects, the level is a nucleic acid level in a biological sample.

In some aspects, the methods further comprise measuring the level of an additional biomarker or a combination of biomarkers shown to correlate with an increased or decreased risk of death and/or an increased or decreased risk of an improvement or a decline in physical health. The disclosure includes the use of one or more of these biomarkers in methods for determining risk or mortality, methods of improving health or decreasing risk of mortality, methods of monitoring the efficacy or success of a therapeutic treatment over a period of time, and/or methods of determining the efficacy of a therapeutic treatment in decreasing risk of mortality or decreasing risk of poor health in a subject or patient. The terms “mortality risk” and “risk of mortality”, as used herein, are used interchangeably.

In some aspects, the methods of the disclosure comprise treating a subject. As used herein, the terms “treat”, “treating”, and “treatment” refer to therapeutic treatment, including prophylactic or preventative measures, wherein the object is to prevent or slow down (lessen) an undesired physiological change associated with a disease or disorder. Beneficial or desired clinical results include, but are not limited to, alleviation of symptoms, diminishment of the extent of a disease or disorder prior to other treatments, such as surgery, stabilization of a disease or disorder (i.e., where the disease or disorder does not worsen), delay or slowing of the progression of a disease or disorder, amelioration or palliation of the disease or disorder, and remission (whether partial or total) of the disease or disorder, whether detectable or undetectable, and prevention of disease or disorder recurrence. “Treatment”, in some aspects, also means prolonging survival as compared to expected survival if not receiving treatment. Those in need of treatment include those already with the disease or disorder, those prone to or at risk of having the disease or disorder, or those in which the disease or disorder is to be prevented.

In some aspects, the methods of the disclosure are associated with predicting risk of mortality from a disease or disorder, or predicting all-cause mortality. The disclosure includes predicting risk of mortality from any disease or disorder. In some aspects, the methods of the disclosure include treating a disease or disorder with a therapeutic agent and determining the effect of the therapeutic agent on the risk of mortality or in improving health of the subject. Thus, in some aspects, the methods of the disclosure are associated with monitoring risk of mortality from a disease or disorder, or monitoring risk of all-cause mortality over a period of time, especially over a period of time involving treatment of the disease or disorder. In some aspects, the disease or disorder includes, but is not limited to, a neoplasia (including cancer), a nervous system disease or disorder, a circulatory disease or disorder, a respiratory disease or disorder, an inflammatory disease or disorder, or an autoimmune disease or disorder.

In some embodiments, the disclosure comprises a method for treating a neoplasm or neoplasia. The term “neoplasia,” as used herein, refers to abnormal or uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

In some aspects, the neoplasia is a solid tumor. In some aspects, the neoplasia is not a solid tumor. In some aspects, the neoplasia is associated with a lesion. In some aspects, the lesion is a pre-malignant lesion. In some aspects, the lesion is a normal tissue at a risk of transforming into malignancy. In some aspects, the lesion is hidden or is an undetected lesion. In some aspects, the lesion is malignant and is a cancer. In some aspects, the neoplasia or cancer is present in the adrenal gland, anus, auditory nerve, bile duct, bladder, bone, brain, breast, central nervous system, cervix, colon, ear, endometrium, esophagus, eye, eyelids, fallopian tube, gastrointestinal tract, head and neck, heart, kidney, larynx, liver, lung, mandible, mandibular condyle, maxilla, mouth, nasopharynx, nose, oral cavity, ovary, pancreas, parotid gland, penis, pinna, pituitary, prostate gland, rectum, retina, salivary gland, skin, small intestine, spinal cord, stomach, testes, thyroid, tonsil, urethra, uterus, vagina, vestibulocochlear nerve and vulva neoplasms, lymph, or lymph node.

In some embodiments, the disclosure comprises a method for treating a nervous system disorder or disease or determining a subject's risk of mortality from a nervous system disorder or disease. In some aspects, the nervous system disorder or disease includes, but is not limited to, Alzheimer's disease, Bell's palsy, cerebral palsy, epilepsy, motor neurone disease (MND), multiple sclerosis (MS), neurofibromatosis, Parkinson's disease, amyotrophic lateral sclerosis (ALS), Huntington's disease, peripheral neuropathies, sciatica, or shingles.

In some embodiments, the disclosure comprises a method for treating a circulatory disorder or disease or determining a subject's risk of mortality from a circulatory disorder or disease. In some aspects, the circulatory disorder or disease, or circulatory system disorder or disease includes, but is not limited to, cardiovascular disease, such as hypertension, hyperlipidemia, hypercholesterolemia, peripheral artery disease, venous thromboembolism, aortic aneurysm, atherosclerosis, improper vascular function, dyslipidemia, stenosis, restenosis, myocardial infarction, cardiac ischemia, arrhythmia and dysrhythmia, angina pectoris, mitral stenosis, mitral valve prolapse, mitral valve regurgitation, stroke, intracranial hemorrhage, acute coronary syndrome, heart failure, stable angina pectoris, or unstable angina pectoris. In some aspects, the disorder is kidney disease resulting from cardiovascular disease.

In some embodiments, the disclosure comprises a method for treating a respiratory disorder or disease or determining a subject's risk of mortality from a respiratory disorder or disease. In some aspects, treating the respiratory disorder or disease comprises reducing a state of inflammation. In some aspects, the respiratory disorder or disease is an inflammatory condition. In some aspects, the respiratory disorder or disease is an airway disease. In some aspects, the respiratory disorder or disease is a cardiac, vascular, or pulmonary disorder. In some aspects, the respiratory disorder or disease includes, but is not limited to asthma, allergy, carbon monoxide poisoning, smoke inhalation, emphysema, asbestos poisoning, bronchitis, pulmonary fibrosis, cystic fibrosis, embolism, Chronic Obstructive Pulmonary Disease (COPD), adult respiratory distress syndrome, pulmonary hypertension, Celiac's disease, or pneumonitis.

In some embodiments, the disclosure comprises a method for treating inflammation or an inflammatory disorder or disease or determining a subject's risk of mortality from an inflammatory disorder or disease. Inflammation represents a fundamental mechanism of diseases caused by microbial, autoimmune, metabolic, and physical insults.

Inflammatory disorders include, but are not limited to, acute pancreatitis; amyotrophic lateral sclerosis (ALS, or Lou Gehrig's disease); Alzheimer's disease; cachexia/anorexia, including, but not limited to, AIDS-induced cachexia; asthma and other pulmonary diseases; atherosclerosis; autoimmune vasculitis; chronic fatigue syndrome; Clostridium associated illnesses, including, but not limited to, Clostridium-associated diarrhea; coronary conditions and indications, including, but not limited to, congestive heart failure, coronary restenosis, myocardial infarction, myocardial dysfunction (e.g., related to sepsis), and coronary artery bypass graft; cancer, including, but not limited to, leukemias, including, but not limited to, multiple myeloma leukemia and myelogenous (e.g., AML and CML), and tumor metastasis; diabetes (including, but not limited to, insulin-dependent diabetes); endometriosis; fever; fibromyalgia; glomerulonephritis; graft versus host disease and/or transplant rejection; hemohorragic shock; hyperalgesia; inflammatory bowel disease; inflammatory conditions of a joint, including, but not limited to, osteoarthritis, psoriatic arthritis, and rheumatoid arthritis; inflammatory eye disease, including, but not limited to, those associated with, for example, corneal transplant; ischemia, including, but not limited to, cerebral ischemia (including, but not limited to, brain injury as a result of, e.g., trauma, epilepsy, hemorrhage or stroke, each of which may lead to neurodegeneration); Kawasaki's disease; learning impairment; lung diseases (including, but not limited to, acute respiratory distress syndrome, or ARDS); multiple sclerosis; myopathies (e.g., muscle protein metabolism, including, but not limited to, muscle protein metabolism in sepsis); neurotoxicity (including, but not limited to, such condition induced by HIV); osteoporosis; pain, including, but not limited to, cancer-related pain; Parkinson's disease; periodontal disease; pre-term labor; psoriasis; reperfusion injury; septic shock; side effects from radiation therapy; temporal mandibular joint disease; sleep disturbance; uveitis; and an inflammatory condition resulting from, e.g., strain, sprain, cartilage damage, trauma, orthopedic surgery, infection, or other disease processes.

In some embodiments, the disclosure comprises a method for treating an autoimmune disorder or disease or determining a subject's risk of mortality from an autoimmune disorder or disease. Autoimmune disorders include, but are not limited to, rheumatoid arthritis, systemic lupus erythematosus, inflammatory bowel disease, multiple sclerosis, type 1 diabetes mellitus, Guillain-Barre syndrome, chronic inflammatory demyelinating polyneuropathy, immune (idiopathic) thrombocytompenic purpura (ITP), psoriasis, Graves' disease, Hashimoto's thyroiditis, myasthenia gravis, vasculitis, scleroderma, and Sjogren's syndrome.

In some embodiments, the disclosure comprises methods for treating a disease or disorder associated with an increased risk of mortality or an increased risk of poor health. “An increased risk of mortality”, as used herein, is a measure of an increased likelihood of death for a subject or patient. Likewise, “a decreased risk of mortality”, as used herein is a measure of a decreased likelihood of death for a subject or patient. “Poor health” or as used herein, is a condition of inferior health in which some disease or impairment of function is present. “Improved health”, as used herein, is a measure of a level of improvement in the condition of inferior health or a level in improvement in the impairment of function from which the subject or patient suffers. “Declining health”, as used herein, is a measure of a level of further impairment in the condition of inferior health or a level of further impairment of function from which the subject or patient suffers.

In some embodiments, the disclosure comprises therapeutic intervention or a therapeutic agent for treating a disease or disorder associated with an increased risk of mortality or an increased risk of poor health. In some embodiments, the disclosure includes the use of therapeutic intervention to lower the mortality risk of a subject. In some aspects, the therapy or therapeutic treatment or intervention is a change in diet, fitness, or other lifestyle changes. In some aspects, the therapeutic agent for treating a disease or disorder is any one or more of Aimovig™ (erenumab-aooe; AMG 334), Aranesp® (darbepoetin alfa), AVSOLA™ (infliximab-axxq), BLINCYTO® (blinatumomab), Corlanor® (ivabradine), Enbrel® (etanercept), EPOGEN® (epoetin alfa), EVENITY™ (romosozumab-aqqg), IMLYGIC® (talimogene laherparepvec), Kanjinti™ (trastuzumab-anns), Kyprolis® (carfilzomib), MVASI™ (bevacizumab-awwb), Neulasta® (pegfilgrastim), NEUPOGEN® (filgrastim), Nplate® (romiplostim), Otezla® (apremilast), Parsabiv™ (etelcalcetide), Prolia® (denosumab), Repatha® (evolocumab), Sensipar® (cinacalcet), Vectibix® (panitumumab), or XGEVA® (denosumab).

In some aspects, the therapeutic agent is Aimovig™ (erenumab-aooe; AMG 334) for treating migraine headaches. In some aspects, the therapeutic agent is Aranesp® (darbepoetin alfa) or EPOGEN® (epoetin alfa) for treating anemia, which in some aspects is associated with chronic renal failure, including patients on dialysis and patients not on dialysis, and for the treatment of anemia in patients with non-myeloid malignancies where anemia is due to the effect of concomitantly administered chemotherapy. In some aspects, the therapeutic agent is AVSOLA™ (infliximab-axxq) for treating autoimmune disease or disorder. In some aspects, the therapeutic agent is BLINCYTO® (blinatumomab) for treating Philadelphia chromosome-negative precursor B-cell acute lymphoblastic leukemia (B-cell ALL). In some aspects, the therapeutic agent is Corlanor® (ivabradine) for treating coronary artery disease and chronic heart failure. In some aspects, the therapeutic agent is Enbrel® (etanercept) for treating inflammatory and immune diseases. In some aspects, the therapeutic agent is EVENITY™ (romosozumab-aqqg) for treating postmenopausal osteoporosis and fracture healing. In some aspects, the therapeutic agent is IMLYGIC® (talimogene laherparepvec) for treating tumors by causing lysis of tumors, followed by release of tumor-derived antigens, which together with virally derived GM-CSF may promote an antitumor immune response. In some aspects, the therapeutic agent is Kanjinti™ (trastuzumab-anns) for treating breast or gastric cancers. In some aspects, the therapeutic agent is Kyprolis® (carfilzomib) for treating multiple myeloma. In some aspects, the therapeutic agent is MVASI™ (bevacizumab-awwb) for treating cancer. In some aspects, the therapeutic agent is Neulasta® (pegfilgrastim) or NEUPOGEN® (filgrastim) for treating subjects with cancer undergoing chemotherapy to decrease the incidence of infection, by treating neutropenia, a lack of certain white blood cells caused by receiving cancer chemotherapy. In some aspects, the therapeutic agent is Nplate® (romiplostim) for treating immune (idiopathic) thrombocytompenic purpura (ITP). In some aspects, the therapeutic agent is Otezla® (apremilast) for treating various inflammatory diseases. In some aspects, the therapeutic agent is Parsabiv™ (etelcalcetide) for treating secondary hyperparathyroidism (sHPT) in patients with chronic kidney disease (KD) on hemodialysis. In some aspects, the therapeutic agent is Prolia® (denosumab) for treating osteoporosis or to increase bone mass in certain oncology patients undergoing treatment. In some aspects, the therapeutic agent is Repatha® (evolocumab) for treating hypercholesterolemia. In some aspects, the therapeutic agent is Sensipar® (cinacalcet) for treating lower serum calcium concentrations in patients with secondary hyperparathyroidism (HPT), primary HPT, and parathyroid cancer. In some aspects, the therapeutic agent is Vectibix® (panitumumab) for treating cancer. In some aspects, the therapeutic agent is XGEVA® (denosumab) for treating bone diseases characterized by excessive bone resorption, including those from cancer-related bone destruction, treatment-related bone loss, and pathologic bone loss.

In some aspects, the disclosure provides a method of treating a subject to reduce risk of mortality of the subject, the method comprising:
measuring the level of a protein in a biological sample from the subject;
comparing the measured level of the protein to a reference level for the protein, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSF1A);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2); and
administering to the subject an effective dose of a therapeutic treatment to decrease the risk of mortality of the subject if
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level and/or;
   (ii) the level of ANTRX2, HPGDS, ADAM22, GHR, or CILP2 is decreased relative to the reference level.

In some aspects, the disclosure provides a method for determining the efficacy of a therapeutic treatment in a subject to reduce risk of mortality of a subject, the method comprising:
measuring the level of a protein in a biological sample from the subject before the therapeutic treatment, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSF1A);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2);
measuring the level of the protein in a biological sample from the subject after administering the treatment; and
determining that the treatment is effective in reducing risk of mortality of the subject if
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is decreased relative to the reference level or to the level in the sample from the subject before treatment; and/or
   (ii) the level of ANTRX2, HPGDS, ADAM22, GHR, or CILP2 is increased relative to the reference level or to the level in the sample from the subject before treatment.

In some aspects, the disclosure provides a method for predicting increased risk of mortality of a subject, the method comprising:
measuring the level of a protein in a biological sample from the subject and comparing the level to a reference level, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2),
wherein the subject is determined to have an increased risk of mortality when
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level; and/or
   (ii) the level of ANTRX2, HPGDS, ADAM22, GHR, or CILP2 is decreased relative to the reference level.

In some aspects, the methods of the disclosure comprise measuring the level of each of any two or more of
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; and/or
(at) WNT1-inducible-signaling pathway protein 2 (WISP-2); and
comparing the measured level of each of the proteins to the reference level for each of the proteins, wherein the subject is determined to have an increased risk of mortality when
   (i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level; and/or
   (ii) the level of ANTRX2, HPGDS, ADAM22, GHR, or CILP2 is decreased relative to the reference level.

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) anthrax toxin receptor 2 (ANTRX2).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-2 (SPON2);
(h) serum amyloid A-1 protein (SAA1);
(i) serum amyloid A-2 protein (SAA2); and/or
(j) C5a anaphylatoxin (C5aAT).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSFiA);
(h) angiopoietin-2 (ANG2);
(i) macrophage metalloelastase (MMP12); and/or
(j) spondin-2 (SPON2).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSF1A);
(h) angiopoietin-2 (ANG2);
(i) macrophage metalloelastase (MMP12); and/or
(j) transgelin (TAGLN).

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) angiopoietin-2 (ANG2);
(g) tumor necrosis factor receptor superfamily member 1A (TNFRSF1A);
(h) macrophage metalloelastase (MMP12);
(i) sushi, von Willebrand factor type A, EGF and pentraxin domain-containing protein 1 (SVEP1); or
(j) C5a anaphylatoxin (C5aAT).

In some aspects, the protein and/or proteins is
(a) growth/differentiation factor 15 (GDF15);
(b) thrombospondin-2;
(c) retinoblastoma-like protein 2;
(d) WAP four-disulfide core domain protein 2;
(e) anthrax toxin receptor 2;
(f) alpha-1-antichymotrypsin complex;
(g) spondin-2;
(h) serum amyloid A-1;
(i) serum amyloid A-2;
(j) C5a anaphylatoxin;
(k) tumor necrosis factor receptor superfamily member 1A;
(l) angiopoietin-2;
(m) macrophage metalloelastase;
(n) transgelin; and/or
(o) sushi, von Willebrand factor type A, EGF and pentraxin domain-containing protein 1.

In some aspects, the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) alpha-1-antichymotrypsin complex (ACT COMPLEX or SERPINA3).

In some aspects, the protein and/or proteins is
(a) hematopoietic prostaglandin D synthase (HPGDS);
(b) anthrax toxin receptor 2 (ANTRX2);
(c) disintegrin and metalloproteinase domain-containing protein 22 (ADAM22); (d) growth hormone receptor (GHR); and/or
(e) insulin-like growth factor-binding protein 2 (IGFPB2).

In some aspects, the protein and/or proteins is
(a) growth/differentiation factor 15 (GDF15);
(b) WAP four-disulfide core domain protein 2 (WFDC2);
(c) cardiac troponin T (cTnT);
(d) cartilage intermediate layer protein 2 (CILP2); and/or
(e) hematopoietic prostaglandin D synthase (HPGDS).

In some aspects, the protein and/or proteins is
(a) growth/differentiation factor 15 (GDF15);
(b) WAP four-disulfide core domain protein 2 (WFDC2);
(c)N-terminal pro-BNP (NTproBNP);
(d) transmembrane emp24 domain-containing protein 10 (TMED10); and/or
(e) pancreatic ribonuclease (RNase1).

In some aspects, the protein and/or proteins is
(a) growth/differentiation factor 15 (GDF15);
(b) tyrosine-protein kinase transmembrane receptor ROR2 (ROR2);
(c) WAP four-disulfide core domain protein 2 (WFDC2);
(d) beta-2-microglobulin (beta-2-M); and/or
(e) tumor necrosis factor receptor superfamily member 1A (TNFRSF1A).

In some aspects, wherein when the subject is predicted to have an increased risk of mortality, the methods of the disclosure further comprise administering a therapeutic treatment to the subject to reduce the increased risk.

In cases in which a method comprises combination of steps, each and every combination or sub-combination of the steps is encompassed within the scope of the disclosure, unless otherwise noted herein.

In regard to any of the methods provided, the steps of the method may occur simultaneously or sequentially. When the steps of the method occur sequentially, the steps may occur in any order, unless noted otherwise.

In some aspects, the subject or patient is suffering from or is at risk of suffering from a neoplasm or other proliferative disease. In some aspects, the subject or patient is suffering from or is at risk of suffering from a nervous system disorder or disease. In some aspects, the subject or patient is suffering from or is at risk of suffering from a circulatory disorder or disease. In some aspects, the subject or patient is suffering from or is at risk of suffering from a respiratory disease or disorder. In some aspects, the subject or patient is suffering from or is at risk of suffering from inflammation or an inflammatory disorder or disease. In some aspects, the subject or patient is suffering from or is at risk of suffering from an autoimmune disorder or disease.

In some aspects, the therapeutic agent is a biological or a pharmaceutical drug that is useful in treating a neoplasm or neoplasia including, but not limited to, cancer, a nervous system disorder or disease, a circulatory system disorder or disease, a respiratory disorder or disease, an inflammatory disorder or disease, or an autoimmune disorder or disease.

In some aspects, therefore, the therapeutic agent or treatment is any one or more of Aimovig™ (erenumab-aooe; AMG 334), Aranesp® (darbepoetin alfa), AVSOLA™ (infliximab-axxq), BLINCYTO® (blinatumomab), Corlanor® (ivabradine), Enbrel® (etanercept), EPOGEN® (epoetin alfa), EVENITY™ (romosozumab-aqqg), IMLYGIC® (talimogene laherparepvec), Kanjinti™ (trastuzumab-anns), Kyprolis® (carfilzomib), MVASI™ (bevacizumab-awwb), Neulasta® (pegfilgrastim), NEUPOGEN® (filgrastim), Nplate® (romiplostim), Otezla® (apremilast), Parsabiv™ (etelcalcetide), Prolia® (denosumab), Repatha® (evolocumab), Sensipar® (cinacalcet), Vectibix® (panitumumab), or XGEVA® (denosumab).

In some aspects, Aimovig™ (erenumab-aooe; AMG 334) is used for treating migraine headaches. In some aspects, Aranesp® (darbepoetin alfa) or EPOGEN® (epoetin alfa) is used for treating anemia including, but not limited to, anemia associated with chronic renal failure, including patients on dialysis and patients not on dialysis, and for the treatment of anemia in patients with non-myeloid malignancies where anemia is due to the effect of concomitantly administered chemotherapy. In some aspects, AVSOLA™ (infliximab-axxq) is used for treating autoimmune disease or disorder. In some aspects, BLINCYTO® (blinatumomab) is used for treating Philadelphia chromosome-negative precursor B-cell acute lymphoblastic leukemia (B-cell ALL). In some aspects, Corlanor® (ivabradine) is used for treating coronary artery disease and chronic heart failure. In some aspects, Enbrel® (etanercept) is used for treating inflammatory and immune diseases. In some aspects, EVENITY™ (romosozumab-aqqg) is used for treating postmenopausal osteoporosis and fracture healing. In some aspects, IMLYGIC® (talimogene laherparepvec) is used for treating tumors by causing lysis of tumors, followed by release of tumor-derived antigens, which together with virally derived GM-CSF may promote an antitumor immune response. In some aspects, Kanjinti™ (trastuzumab-anns) is used for treating breast or gastric cancers. In some aspects, Kyprolis® (carfilzomib) is used for treating multiple myeloma. In some aspects, MVASI™ (bevacizumab-awwb) is used for treating cancer. In some aspects, Neulasta® (pegfilgrastim) or NEUPOGEN® (filgrastim) is used for treating subjects with cancer undergoing chemotherapy to decrease the incidence of infection, by treating neutropenia, a lack of certain white blood cells caused by receiving cancer chemotherapy. In some aspects, Nplate® (romiplostim) is used for treating immune (idiopathic) thrombocytompenic purpura (ITP). In some aspects, Otezla® (apremilast) is used for treating various inflammatory diseases. In some aspects, Parsabiv™ (etelcalcetide) is used for treating secondary hyperparathyroidism (sHPT) in patients with chronic kidney disease (KD) on hemodialysis. In some aspects, Prolia® (denosumab) for treating osteoporosis or to increase bone mass in certain oncology patients undergoing treatment. In some aspects, Repatha® (evolocumab) is used for treating hypercholesterolemia. In some aspects, Sensipar® (cinacalcet) is used for treating lower serum calcium concentrations in patients with secondary hyperparathyroidism (HPT), primary HPT, and parathyroid cancer. In some aspects, Vectibix® (panitumumab) is used for treating cancer. In some aspects, XGEVA® (denosumab) is used for treating bone diseases characterized by excessive bone resorption, including those from cancer-related bone destruction, treatment-related bone loss, and pathologic bone loss.

In some aspects, the therapeutic agent is a biological or pharmaceutical drug. In some aspects, such a biological or pharmaceutical drug is a statin. In some aspects the statin is atorvastatin (Lipitor®), fluvastatin (Lescol®), lovastatin, pitavastatin (Livalo®), pravastatin (Pravachol®), rosuvastatin calcium (Crestor®), or simvastatin (Zocor®).

In some aspects, the therapeutic treatment is a dietary restriction, a fitness regimen, or the supplementation of diet.

The disclosure also includes kits which comprise reagents packaged in a manner which facilitates their use for measuring a biomarker in a biological sample from a subject. In some variations, such reagents are packaged together. In some variations, the kit further includes an analysis tool for evaluating the probability that the subject will favorably respond to a therapeutic treatment after taking a measurement of at least one biomarker from a biological sample from the subject.

In one embodiment, the disclosure pertains to a kit for assaying a sample from a subject to determine the likelihood that the patient will positively respond to a therapeutic treatment, wherein the kit comprises reagents necessary for selectively detecting the relative level or the absolute level of the biomarker or a combination of biomarkers in the subject and comparing them to a reference level of the biomarker. In certain embodiments, the biomarker is at least one or a combination of any one or more of the biomarkers disclosed herein. In certain embodiments, the kit comprises one or more reagents for detecting and/or measuring the relative expression level or the absolute expression level of any one of the biomarkers disclosed herein, or a combination of any two or more thereof in a sample from a subject.

In a specific embodiment, the kits of the disclosure each contain an apparatus for collecting a biological sample from a subject and reagents for measuring the level of biomarker in a biological sample. In a further aspect, the kit comprises optional instructions included in the package that describes use of the reagents packaged in the kit for practicing the method.

In a further aspect, the disclosure includes a pharmaceutical pack (kit), the pack comprising a therapeutic agent and a set of instructions for administration of the therapeutic agent to a subject diagnostically tested and determined to be a subject who will favorably respond to treatment with the therapeutic agent. The therapeutic agent can be any of the therapeutic agents described herein.

In some embodiments, the kit further comprises a set of instructions for using the reagents comprising the kit. In certain embodiments, the kit further comprises a collection of data comprising correlation data between the biomarker level and the probability that the subject will favorably respond to the therapeutic treatment. In some aspects, therefore, the kit provides a means for measuring the relative level of the biomarker or biomarkers and determining the relative increase or decrease in the level of the biomarker from a sample from the subject compared to the reference level, or the level of the biomarker before treatment.

All methods described herein are performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the methods of the disclosure.

When used herein “consisting of” excludes any element, step, or ingredient not specified in the claim element. When used herein, “consisting essentially of” does not exclude materials or steps that do not materially affect the basic and novel characteristics of the claim. In each instance herein, any of the terms “comprising”, “consisting essentially of” and “consisting of” may be replaced with either of the other two terms.

The above description and the below examples provide exemplary arrangements, but the disclosure is not limited to the specific methodologies, techniques, protocols, material, reagents, substances, etc., described herein and as such can vary. The terminology used herein serves to describe specific embodiments only. The terminology used herein does not intend to limit the scope of the disclosure, which is defined solely by the claims. Aspects of the disclosure are provided in the independent claims. Some optional features of are provided in the dependent claims.

Each publication, patent application, patent, and other reference cited herein is incorporated by reference in its entirety to the extent that it is not inconsistent with the present disclosure.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

Materials and Methods

Study Populations

The participants were all Icelandic, and the plasma samples were collected at two time periods. In the first data set, 22,913 participants were recruited in the years 2000-2006 at deCODE (Reykjavik, Iceland) through an Icelandic cancer project (ICP) (N=20,226) and various smaller projects (VSP1) (N=2,687). The second dataset consists of participants recruited through the deCODE health study (DHS) (N=8,814) in the years 2016-2019, and participants recruited in various smaller projects (VSP2) (N=6,798) at deCODE in the years 2010-2019. Since very little follow up (mean 1.4 years, sd. 1.1 years) was available for the 2010-2019 samples, only the samples from 2000-2006 (mean follow up 13.7 years, sd. 4.7 years) were used for the development of models to predict both long-term and short-term all-cause mortality. Deaths and the causes of death until the end of 2018 were obtained from the Icelandic death registry. The Icelandic prescription registry provided medication information, while the Icelandic health care system gave information on the prevalence of type 2 diabetes (T2D), coronary artery disease (CAD), myocardial infarction (MI), cancer, and all other baseline information.

Pregnant women (N=145), individuals with deaths from external causes (ICD10 S00-T98, N=241), and participants younger than 18 were excluded from the study.

The cause of death was documented with ICD-10 codes. Codes C00-D48 are connected to neoplasms, G00-G99 to the nervous system, 100-199 to the circulatory system, J00-J99 to the respiratory system, and all other codes were taken as one category of other causes.

All participants who donated samples gave informed consent, and the National Bioethics Committee of Iceland approved the study, which was conducted in agreement with conditions issued by the Data Protection Authority of Iceland (VSN_14-015, VSN_15-130, and VSN_15-214). Personal identities of the participant's data and biological samples were encrypted by a third-party system (Identity Protection System), approved and monitored by the Data Protection Authority.

Protein Measurements

Blood was collected in EDTA tubes. The tubes were inverted 4-5 times before being centrifuged for 10 min at 3000 g at 4° C. Plasma samples were frozen in aliquots at −80° C. Plasma aliquots were kept away from light while they were allowed to thaw on ice. The aliquots were mixed by inverting the tubes three times and then centrifuged for 10 min at 3220 g at 4° C. before measurement.

All samples were measured with the SomaScan® platform (SomaScan® Proteomics) (https-colon-forward slash-forward slash-www.somalogic.com) (SomaLogic®, Boulder, CO), containing 5,284 aptamers providing measurements of the relative binding of the plasma sample to each of the aptamers in relative fluorescence units (RFU). As a quality control, the correlation of log-transformed RFU units over all the 5,284 aptamers for every pair of samples was calculated. The average correlation of each sample with all other samples was then calculated. The average correlation was high (median=0.94), and samples with a correlation of less than 0.82 were excluded. Furthermore, for evaluating the internal repeatability of the SOMAscan platform, 200 samples drawn from the same individuals at different time points and 228 that were replicates of the same sample were examined. The replicates were used to exclude aptamers that were not robust within the same sample. Aptamers that were not measuring human proteins were also excluded, resulting in a total of 4,905 protein aptamers. To maintain consistency in the dataset, the data was restricted to one sample per person. In the case of repeated measurements on the same individual, the newest sample was chosen, and in the case of replicated measurements of the same sample, one measurement was selected at random.

Additionally, protein measurements were carried out with the Olink® Explore platform (Olink Proteomics, Uppsala, Sweden). Protein measurements, in various aspects, are carried out on any other proteomics platforms and assays known in the art and are not limited to SomaScan® or Olink® Explore platforms.

All protein levels were log-transformed. The ICP and VSP1 data were randomly split 70%/30% into training/test sets. The means and standard deviations of the training set were used to standardize all features used for prediction. Only the training set was used for feature and model selection.

Features of the Development Set

If possible, all features were recorded/collected at the time of plasma collection. Age, age squared, sex, and their interactions were included in all models. The baseline model also had current smoking status, T2D, CAD, previous MI, previous cancer diagnosis, use of statins, hypertension treatment, BMI, BMI squared, ApoB, and ApoB x statin. Most of the BMI values were available at the time of plasma collection, but 1832 were imputed with the median of all available BMI measurements for that individual, and BMI values for 455 individuals with no BMI measurements were imputed with the mean value of training and testing data separately. Since cholesterol levels were only available for a small portion of the participants, the ApoB protein was used as a substitute (Emerging Risk Factors Collaboration et al. Major lipids, apolipoproteins, and risk of vascular disease. JAMA 302, 1993-2000 (2009)). The ApoB protein has a correlation of 0.7 with non-HDL cholesterol in the DHS data, where both are available. Medication information was only available from 2003 and on, so statin use before 2003 was predicted using the proteomics data. Those receiving hypertension treatment in the first half of 2003 with samples collected earlier were assumed to have already been receiving treatment at the time of sample collection. Current smoking status was estimated using the proteomics data when data were not available.

Additionally, baseline variable interactions with age and sex that had a p-value lower than 0.1 in logistic regression models for death within one, five, ten, or fifteen years were included in the baseline model. Those were BMI, BMI squared, CAD, MI, cancer, statin use, and hypertension treatment interactions with age and CAD, smoking, and diabetes interactions with sex.

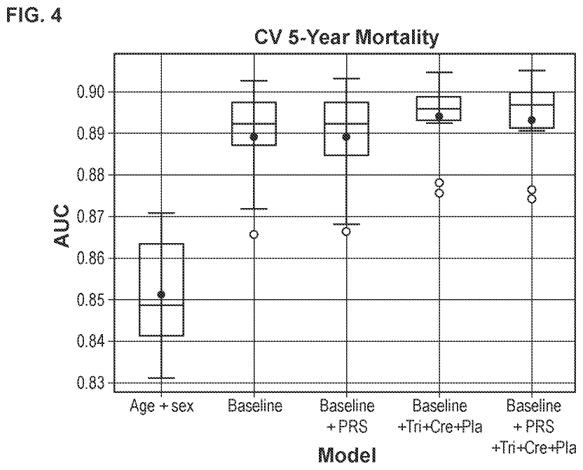

Other baseline features considered were creatinine levels, platelet numbers, platelet numbers squared, and triglycerides levels. Even though these factors did contribute to the baseline prediction, they were not included in the final baseline model due to many missing values and concerns that these measurements were more recent than the protein measurements (FIG. 4).

Polygenic risk scores for cancer, hypertension, stroke, CAD, Alzheimer's, attention deficit hyperactivity disorder (ADHD), Parkinson's, educational attainment, depression, bipolar disorder, BMI, schizophrenia, IQ, autism, and anorexia were tried as features in the all-cause mortality risk predictor. They did not improve the baseline prediction and were therefore not used any further (FIG. 4).

The Boruta feature selection method (Kursa, M. B. & Rudnicki, W. R. Feature Selection with the Boruta Package. JSS J. Stat. Softw. 36, (2010)) was used to select all relevant proteins out of 4,905. This was done separately for events within 1, 2, . . . , and 15 years.

Metrics

For comparing prediction performance, the ROC curve, the area under the curve (AUC), and the integrated discrimination improvement (IDI) (Pencina, M. J., D'Agostino, R. B., D'Agostino, R. B. & Vasan, R. S. Evaluating the added predictive ability of a new marker: From area under the ROC curve to reclassification and beyond. Stat. Med. 27, 157-172 (2008)) were used. The confidence intervals for the IDI were obtained by bootstrapping. Kaplan-Meier curves with log-log confidence intervals were also examined. Before examining connections to other phenotypes, the predicted values were corrected for age, age squared, sex, and their interactions using linear regression. Calculated correlations are Pearson correlations, and means were compared with a t-test. Significant associations were those with a p-value lower than 0.05 after Bonferroni correction. The quantitative phenotypes were corrected for age and sex and normalized. Calibration was assessed with a visual examination of actual incidence and predicted risk in 5% quantile groups. The test data were used for all model comparisons.

Univariate Associations

Associations of single proteins with mortality were examined using logistic regression. The model included age, age squared, sex, and their interactions as covariates. Significant associations were those with a p-value lower than 0.05 after Bonferroni correction.

Order of Proteins

Two approaches were used to order proteins by effects. The first method was a stepwise forward selection with age, age squared, sex, and their interactions as baseline features, sequentially adding the protein that maximally increased the log-likelihood. The other method trained 1,000 logistic regression models with an L1 penalty each time resampling the training data. The proteins were then ordered by how often they were included in the model. In the case of proteins included equally often, the protein with the higher mean coefficient was ranked higher.

Model Comparison

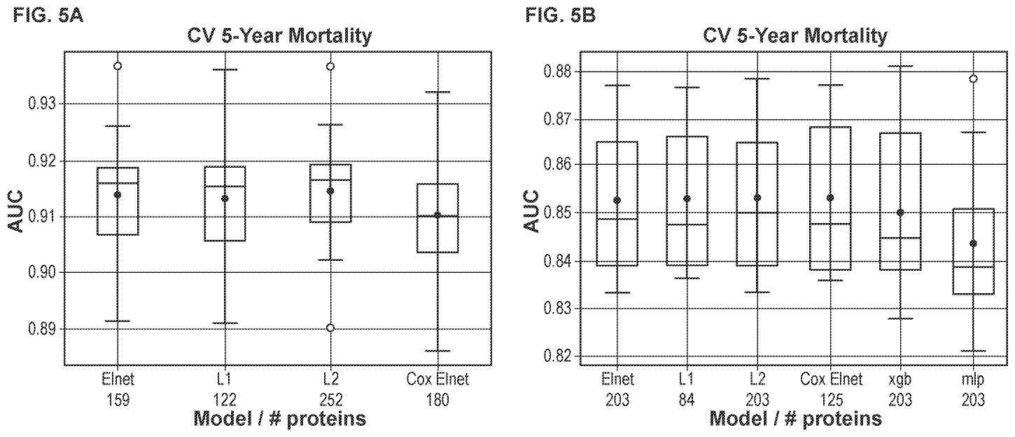

Six different protein prediction models were evaluated for five and ten-year prediction; logistic regression with an L1 penalty, logistic regression with an L2 penalty, logistic regression with an elastic net penalty, multi-layered perceptron (MLP), XGBoost, and a Cox survival model with an elastic net penalty. The parameters for the MLP and XGBoost with a Bayesian optimization algorithm and 5-fold cross-validation (CV) were optimized, while a grid search and 5-fold CV was used for the rest. The parameters were chosen to minimize log-loss except in the Cox models, where the concordance was maximized. The methods were all compared using the mean AUC of 10-fold CV on the training set. Logistic regression performed best, not much better for any penalty type than the other. Logistic regression with an L1 penalty was used for the final model since it used the fewest features (FIG. 5A-B). For the age and sex model, baseline model, and age, sex, and one protein model, logistic regression was used. L2 penalty was added when prediction with age, sex, and 1-100 preselected proteins was performed.

In addition to models trained on all ages, separate models were trained for participants aged 60+ only. All prediction analysis restricted to the 60+ group used predictions by these models.

Other Protein Models

New predictors were trained using logistic regression with PAD or the selected sets of proteins with age, sex, age squared, and their interactions or the baseline as features. Cox proportional hazards models were also experimented with since those models were used in the original predictors, but the logistic regression approach gave better predictions.

To predict biological age using the proteome, we used a linear regression model with L1 penalty. All 4,905 protein measurements and sex were used as candidate features in the model. The penalization strength was selected to minimize the mean square error using 5-fold CV on the training data. The PAD was then calculated as the difference between predicted age and chronological age.

Univariate Associations

Associations of single protein measurements with mortality were examined using logistic regression. The model included age, age squared, sex, and their interactions as covariates. Associations were also examined using Cox proportional hazards models. The Cox model used age, age squared, sex, and their interactions as covariates. To avoid age violating the proportional hazards assumption significantly, the model used different baseline hazards for each age bin; 18-40, 40-60, 60-80, and 80+. Associations were considered significant if they had a p-value lower than 0.05 after Bonferroni correction, i.e., lower than 0.05/4905=1.02e-5. Associations with cause-specific mortality were examined by excluding deaths from all other causes in the data. Correlations of protein measurements were calculated with Pearson correlation after correcting for age, age squared, sex, and their interactions.

Pathway Analysis

For analyzing the over- and underrepresentation of Reactome protein pathways, the PANTHER classification system, version 16 (http-colon-forward slash-forward slash-pantherdb.org/) was used. As a reference, all 4905 protein measurements were used, resulting in 4619 unique Uniprot IDs recognized by the system. In cases where a protein measurement had multiple proteins associated with it, all of them were included. The Fisher exact test was used to determine statistical significance and false discovery rate (FDR) to account for multiple testing. To select proteins relevant to 5-year mortality risk the Boruta feature selection method was used (Kursa et al., Feature selection with the Boruta Package. JSS J. Stat. Softw. 36, 1-13 (2010). To correct for age and sex, linear regression of age, age squared, sex and their interaction were subtracted from the protein levels before applying Boruta.

Ranking of Protein Measurements

Two approaches were used to order protein measurements by effects. The first method was a stepwise forward selection with age, age squared, sex, and their interactions as baseline features, sequentially adding the protein that maximally increased the log-likelihood. The other method trained 1000 logistic regression models with an L1 penalty each time resampling the training data. The protein measurements were then ordered by how often they were included in the model. In the case of protein measurements included equally often, the protein measurement with the higher mean coefficient was ranked higher.

Heritability Estimate

Heritability of five-year predicted mortality risk was estimated by using the correlation of mortality risk between siblings and between parent-offspring pairs. The predictions were corrected for age and sex, normalized and corrected again for age, sex, and year of birth. There were 20,983 sibling pairs and 18,166 parent-offspring pairs in the combined ICP, VSP1, DHS, and VSP2 datasets. Sibling pairs older than 60 years were 7,022, and parent-offspring pairs in this age group were 1,712. The sibling estimated heritability for predicted all-cause mortality risk was 0.22 and 0.24 in each age group and 0.13 and 0.17 when estimated with parent-offspring pairs. This method fails to account for similarities in the environment of relatives, making this an upper bound of heritability.

Associations with Phenotypes

Before examining connections to other phenotypes, the predicted values were corrected for age, age squared, sex, and their interactions using linear regression. Calculated correlations are Pearson correlations, and means were compared with a two-sided t-test. Associations were considered significant if they had a p-value lower than 0.05 after Bonferroni correction. The quantitative phenotypes were corrected for age and sex and normalized.

Statistics, Reproducibility, and Software

Standard metrics and statistical tests were used to evaluate and compare models. All data preparations, plotting, and model training were done in Python version 3.6.3 (Python Software Foundation). Some metrics were calculated in R version 3.6.0 using the Python library rpy2 (Python Software Foundation). The logistic regression models were implemented using the machine learning library scikit-learn (Pedregosa et al., J. Mach. Learn. Res. 12, 2825-2830 (2011)). AUCs were compared using R version 3.6.0 with the package pROC (Robin et al., BMC Bioinform. 12, 77 (2011)).

### REFERENCES

The protein predictor methods as described herein are further described in Eiriksdottir et al. (“Predicting the probability of death using proteomics.” Commun Biol 4, 758 (2021); https_colon_forward slash_forward slash_doi.org/10.1038/s42003-021-02289-6; “the Eiriksdottir publication”), which is incorporated herein by reference in its entirety. As described in the Eiriksdottir publication, protein measurements are not normalized in the sample set and, therefore, the predictor methods as described herein are used directly on individual protein measurements from, for example, the SomaScan® platform (SomaScan® Proteomics (SomaLogic®), the Olink® Explore platform (Olink Proteomics, Uppsala, Sweden), or any other proteomics platforms and assays known in the art compatible with the system described herein.

### Example 2

Study Results

The ICP and VSP1 model development data included 22,913 participants aged 18-101 (mean 56.6, sd. 17.4) sampled between the years 2000-2006, of which 10,136 were 60 years old or older (mean 73.0, sd. 7.8). The average follow-up time for this group was 13.7 (sd. 4.7) years, until death or the end of the study period at the end of 2018. At the time of sample collection, 7% had CAD, 5.2% previous MI, and 23.9% had been diagnosed with cancer. Since most of this dataset was collected through a cancer project, it has about three times higher cancer prevalence than the more recently collected sample set from the DHS (Table 2).

TABLE 2
Characteristics of all study participants by age and sample sets.

| | ICP + VSP1 | ICP + VSP1 | DHS | DHS | VSP2 | VSP2 |
|---|---|---|---|---|---|---|
| | 18+ | 60+ | 18+ | 60+ | 18+ | 60+ |
| Characteristic | N = 22,913 | N = 10,136 | N = 8,814 | N = 3,684 | N = 6,798 | N = 2,611 |
| Men | 9,991(43.6) | 4,816(47.5) | 3,876(44.0) | 1,652(44.8) | 2,657(39.1) | 1,109(42.5) |
| Women | 12,922(56.4) | 5,320(52.5) | 4,938(56.0) | 2,032(55.2) | 4,141(60.9) | 1502(57.5) |
| Follow up | 13.7(4.7) | 11.1(5.5) | 1.3(0.7) | 1.3(0.7) | 1.6(1.4) | 1.6(1.4) |
| Age | 56.6 (17.4) | 73.0(7.8) | 55.4(14.7) | 69.1(6.4) | 52.9(16.6) | 70.0(6.7) |
| Age-span | 18-101 | 60-101 | 18-96 | 60-96 | 18-98 | 60-98 |
| BMI | 26.5(4.6) | 26.6(4.4) | 28.6(5.3) | 28.9(5.1) | 27.8(5.4) | 27.7(4.9) |
| T2D | 969(4.2) | 809(8.0) | 437(5.0) | 299(8.1) | 258(3.8) | 177(6.8) |
| Statin estimate | 1,897(8.3) | 1,617(16.0) | 1,623(18.4) | 1,256(34.1) | 1,489(21.9) | 1,140(43.7) |
| HT medication | 7,676(33.5) | 5,599(55.2) | 4,137(46.9) | 2,553(69.3) | 3,292(48.4) | 1,962(75.1) |
| Smoker | 2,998(13.1) | 792(7.8) | 844(9.6) | 310(8.4) | 621(9.1) | 235(9.0) |
| CAD | 1,608(7.0) | 1,490(14.7) | 645(7.3) | 570(15.5) | 869(12.8) | 723(27.7) |
| Previous MI | 11,99(5.2) | 1,095(10.8) | 249(2.8) | 202(5.5) | 394(5.8) | 314(12.0) |
| Cancer | 5,484(23.9) | 3,880(38.3) | 675(7.7) | 512(13.9) | 526(7.7) | 400(15.3) |
| diagnosis | | | | | | |
| Deaths | 7,061(30.8) | 6,222(61.4) | 25(0.3) | 12(0.6) | 83(1.2) | 74(2.8) |
| Age at death | 81.2(10.7) | 84.0(7.5) | 76.8(10.1) | 79.8(6.6) | 75.1(10.4) | 77.3(8.5) |
| Cause of death: | | | | | | |
| Neoplasms | 2,687(38.1) | 2,098(33.3) | 12(48.0) | 11(50.0) | 49(59.0) | 43(58.1) |
| Nervous system | 596(8.4) | 550(8.8) | 1(4.0) | 1(4.5) | 3(3.6) | 3(4.1) |
| Circulatory | 2,472(35.0) | 2,345(37.7) | 9(36.0) | 7(31.8) | 23(27.7) | 20(27.0) |
| system | | | | | | |
| Respiratory | 544(7.7) | 507(8.1) | 0(0.0) | 0(0.0) | 6(7.2) | 6(8.1) |
| system | | | | | | |
| Other causes | 762(10.8) | 722(11.6) | 3(12.0) | 3(13.6) | 2(2.4) | 2(2.7) |

The numbers are number(percent of participants), number(percent of total deaths), mean(sd), or range.

During the study period, 7,061 participants (30.8%) died at an average age of 81.2 (sd. 10.7) years. Of those who died, 38.1% of deaths related to neoplasms, 8.4% to the nervous system, 35.0% to the circulatory system, 7.7% to the respiratory system, and 10.8% to other internal causes. Table 3 lists all relevant baseline characteristics. For all participants, measurements of 4,905 proteins were available after a quality check. Models were developed using 70% of the data, and results were reported on the remaining 30%.

TABLE 3
Characteristics of the combined ICP
and VSP1 model development dataset.

| | Older than 18 | Older than 60 |
|---|---|---|
| | N = 22,913 | N = 10,136 |
| Characteristics | Number(%), Mean(SD) | Number(%), Mean(SD) |
| Men | 9,991(43.6) | 4,816(47.5) |
| Women | 12,922(56.4) | 5,320(52.5) |
| Follow up | 13.7(4.7) | 11.1(5.5) |
| Age | 56.6 (17.4) | 73.0(7.8) |
| Age-span | 18-105 | 60-105 |
| BMI | 26.5(4.6) | 26.6(4.4) |
| T2D | 969(4.2) | 809(8.0) |
| Statin use | 1,897(8.3) | 1,617(16.0) |
| HT medication use | 7,676(33.5) | 5,599(55.2) |
| Smoker | 2,998(13.1) | 792(7.8) |
| CAD | 1,608(7.0) | 1,490(14.7) |
| Previous MI | 1,199(5.2) | 1,095(10.8) |
| Cancer diagnosis | 5,484(23.9) | 3,880(38.3) |
| Deaths | 7,061(30.8) | 6,222(61.4) |
| Age at death | 81.2(10.7) | 84.0(7.5) |

| Cause of death: | Number(% of deaths) | Number(% of deaths) |
|---|---|---|
| Neoplasms | 2,687(38.1) | 2,098(33.3) |
| Nervous system | 596(8.4) | 550(8.8) |
| Circulatory system | 2,472(35.0) | 2,345(37.7) |
| Respiratory system | 544(7.7) | 507(8.1) |
| Other causes | 762(10.8) | 722(11.6) |

Prediction Performance at Different Time Points

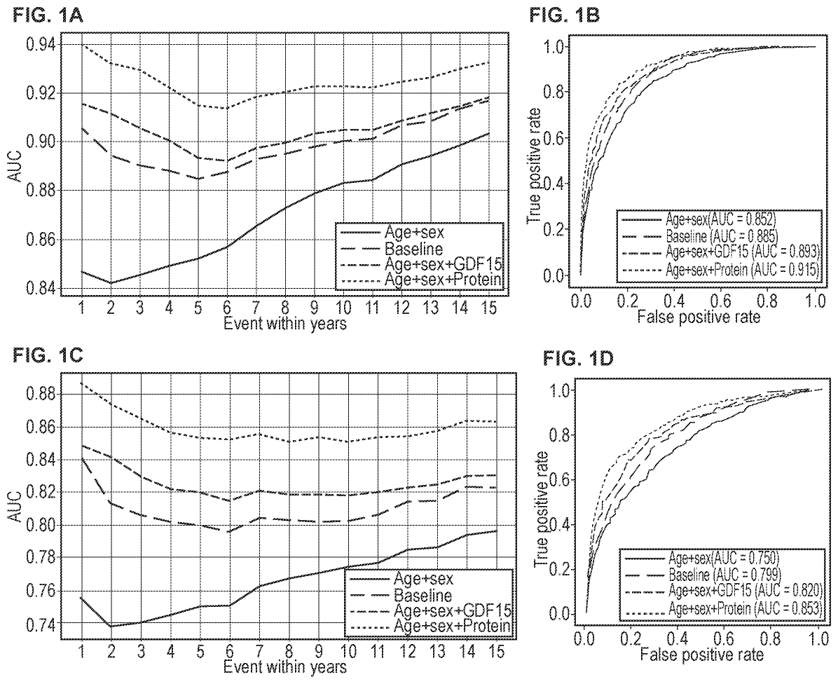

In FIG. 1A-D, the discriminatory power of prediction models is shown, by using a receiver operating characteristics (ROC) curve and the area under the curve (AUC) for participants of all ages (FIG. 1A-B) and restricted to those 60 years or older (FIG. 1C-D).

The AUC for the model based on all participants using only age and sex (age and sex model) yields a good prediction, and the prediction improves with the time from sample collection (FIG. 1A). Adding disease and lifestyle variables to the model (baseline model) yields a considerable improvement over the age and sex model. However, adding the growth/differentiation factor 15 (GDF15) plasma protein, which has the strongest association with all-cause mortality of all 4,905 proteins, to the age and sex model (GDF15 model), yielded a better predictor than the baseline model. Adding more proteins, from the set of 4,905 measured plasma proteins to the age and sex model, gave an even better prediction model (protein model). The difference between the four prediction models was greater for short-term predictions than long-term predictions.

The proteins for the protein model were chosen separately for predictions of all-cause mortality within 1, 2, . . . , 15 years. In general, the protein model needed fewer proteins for short-term predictions than for long-term predictions. The Boruta feature selection chose 209 proteins for prediction of death within one year, but 454 proteins for death within 15 years. The L1 penalty reduced the proteins to a minimum of 81 proteins for prediction of death within one year to a maximum of 219 for death within 13 years. The five-year predictor included 117 proteins, and the ten-year predictor included 199 proteins (Table 4).

TABLE 4
Number of proteins chosen by the Boruta
method and the trained Lasso models.

| Death within | 18+ | 18+ | 60+ | 60+ |
|---|---|---|---|---|
| years | Boruta | Lasso | Boruta | Lasso |
| 1 | 209 | 81 | 177 | 56 |
| 2 | 236 | 98 | 183 | 75 |
| 3 | 236 | 87 | 185 | 75 |
| 4 | 235 | 94 | 182 | 88 |
| 5 | 249 | 117 | 185 | 71 |
| 6 | 263 | 136 | 180 | 93 |
| 7 | 390 | 156 | 215 | 116 |
| 8 | 386 | 193 | 195 | 114 |
| 9 | 407 | 207 | 206 | 106 |
| 10 | 407 | 199 | 214 | 119 |
| 11 | 418 | 207 | 213 | 115 |
| 12 | 423 | 212 | 212 | 116 |
| 13 | 439 | 219 | 220 | 116 |
| 14 | 471 | 190 | 220 | 124 |
| 15 | 454 | 192 | 229 | 115 |

FIG. 1B depicts the ROC-curves for all-cause mortality within five years for all participants. The AUC for the age and sex model was 0.852. The baseline model had an AUC of 0.885, an increase of 0.032 (p-value 4.9e-11) over the age and sex model. The GDF15 model had an AUC of 0.893, with an increase of 0.009 (p-value 0.07) over the baseline model, while the protein model yielded an AUC of 0.915, improving the baseline AUC by 0.03 (p-value 9.2e-10). All AUCs were higher for a ten-year prediction, 0.883, 0.900, 0.905, and 0.922 for the age and sex, baseline, GDF15, and protein models, respectively.

Restricting the analysis to participants 60 years or older lowers the AUCs compared to models including all the participants. However, the differences from the baseline model were greater (FIG. 1C-D). For the five year prediction, the AUCs were 0.750, 0.799, 0.820, and 0.853 for the age and sex, baseline, GDF15, and protein model, respectively. The differences from the baseline were −0.049 (p-value 9.7e-10), 0.020 (p-value 0.02) and 0.054 (p-value 2.7e-9) for the age and sex, GDF15, and protein models, respectively.

For five year prediction, the integrated discrimination improvement (IDI) for the protein model vs. the baseline was 0.116 (95% Cl: 0.096-0.138), and for ten years prediction, it was 0.081 (95% Cl: 0.069-0.093). For 60+ participants the IDI was 0.115 (95% Cl: 0.095-0.137) for five years and 0.092 (95% Cl: 0.078-0.105) for ten years.

Kaplan-Meier Analysis

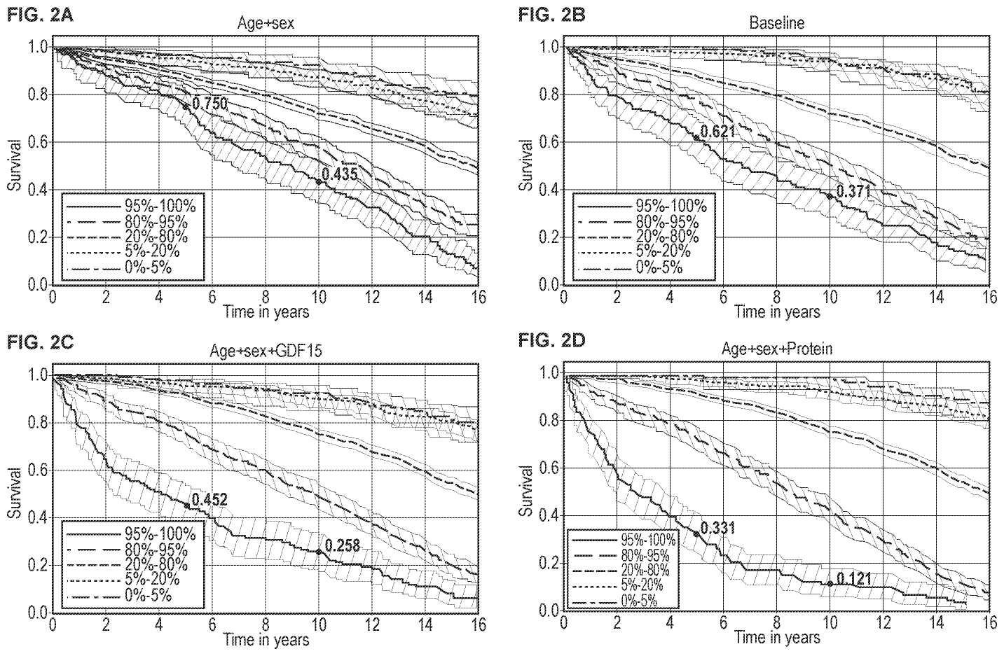

We looked at Kaplan-Meier survival curves for participants in the ICP+ VSP1 test set between 60 and 80 years old to reduce the overwhelming effects of age. That included 2,488 participants, of which 1,312(53.1%) died during the study period, 305(12.6%) within five years, and 701(28.2%) within ten years from sample collection. The curves were plotted separately for the four prediction models. By splitting the Kaplan-Meier curves by quantiles of predicted ten-year risk, the proteins' discriminative power becomes evident (FIG. 2A-D). Of the 5% (124 participants) predicted at the highest risk by the age and sex, baseline, GDF15, and protein model, 25%, 38%, 55%, and 67% died within five years, and 56%, 63%, 74%, and 88% within ten years. Of the 5% (125 participants) predicted at the lowest risk by each model, 8%, 6%, 8%, and 1% died within ten years.

Different Causes of Death

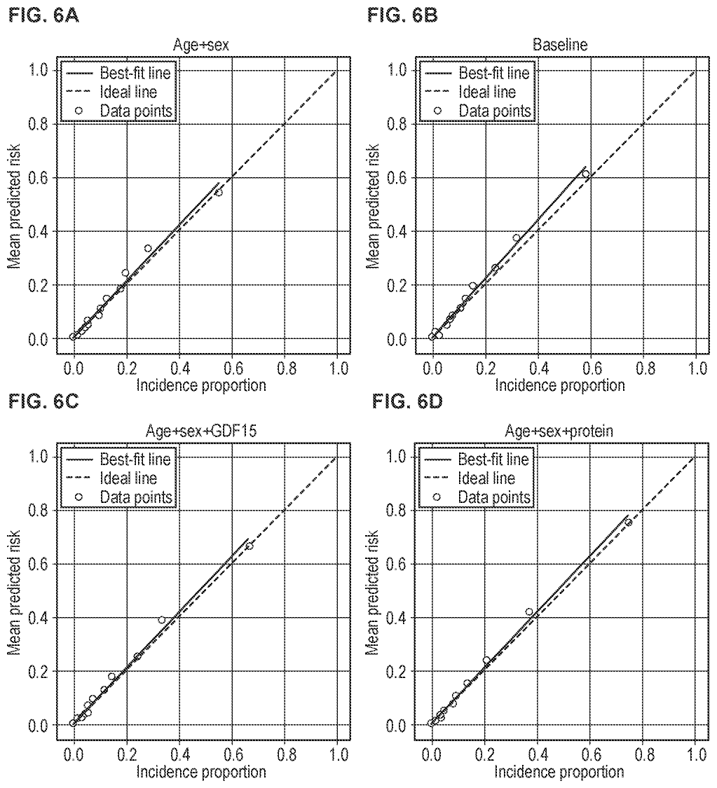

A visual examination showed all models reasonably well-calibrated, allowing predicted risk values to be interpreted directly as probabilities (FIG. 6A-D).

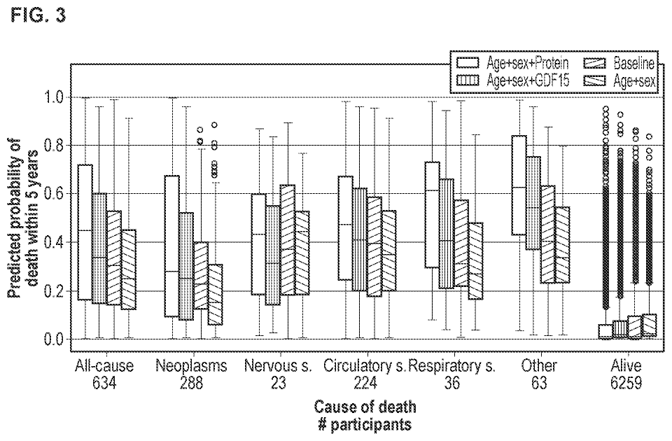

The difference in the performance of the prediction models based on the cause of death was also examined. FIG. 3 shows the predicted five-year risk of all-cause mortality split by survival status after five years from plasma collection. Participants who died within five years are also shown separately for five cause-of-death categories; neoplasms, nervous system, circulatory system, respiratory system, and other. All five cause-of-death categories showed a higher predicted risk for the protein model than the baseline model, although the difference varied between categories. Deaths from neoplasms were not as reliably predicted as deaths from other causes. Deaths from respiratory system causes and other causes showed the greatest improvement of the protein model over the baseline model. The predicted risk for those who did not die within five years was generally lowest with the protein model.

Associations of Individual Proteins

In a univariate association corrected for age and sex, 1,365 proteins were significantly associated with death within five years after a Bonferroni correction. For participants older than 60 years, the number of proteins was 1,069. Of the top ten single protein associations for events within two, five, ten, or fifteen years, six proteins were common for all time points, with GF15 always having the largest effect (Table 5).

TABLE 5
Top ten single protein associations with all-
cause mortality for different time to death.

| Proteins | Beta | P-value |
|---|---|---|
| Death within 2 years | | |
| Growth/differentiation factor 15 | 1.35 | 2.75E−137 |
| Thrombospondin-2 | 0.76 | 8.33E−99 |
| Alpha-1-antichymotrypsin complex | 0.79 | 5.60E−91 |
| Retinoblastoma-like protein 2 | 0.84 | 5.97E−87 |
| WAP four-disulfide core domain protein 2 | 0.92 | 4.62E−85 |
| Spondin-2 | 0.92 | 1.41E−82 |
| Serum amyloid A-1 protein | 0.67 | 8.38E−82 |
| Serum amyloid A-2 protein | 0.54 | 2.26E−80 |
| C5a anaphylatoxin | 0.93 | 1.78E−78 |
| Anthrax toxin receptor 2 | −0.67 | 8.11E−76 |
| Death within 5 years | | |
| Growth/differentiation factor 15 | 1.20 | 1.91E−167 |
| WAP four-disulfide core domain protein 2 | 0.82 | 2.05E−111 |
| Thrombospondin-2 | 0.62 | 9.07E−100 |
| Anthrax toxin receptor 2 | −0.56 | 4.62E−88 |
| Retinoblastoma-like protein 2 | 0.65 | 9.23E−88 |
| Alpha-1-antichymotrypsin complex | 0.61 | 1.51E−86 |
| Tumor necrosis factor receptor superfamily | 0.64 | 9.82E−85 |
| member 1A | | |
| Angiopoietin-2 | 0.64 | 1.11E−82 |
| Macrophage metalloelastase | 0.66 | 3.94E−81 |
| Spondin-2 | 0.67 | 1.40E−80 |
| Death within 10 years | | |
| Growth/differentiation factor 15 | 1.04 | 1.45E−155 |
| WAP four-disulfide core domain protein 2 | 0.76 | 1.01E−116 |
| Macrophage metalloelastase | 0.62 | 1.22E−86 |
| Thrombospondin-2 | 0.50 | 6.10E−81 |
| Angiopoietin-2 | 0.55 | 9.47E−79 |
| Retinoblastoma-like protein 2 | 0.54 | 1.22E−78 |
| Tumor necrosis factor receptor superfamily | 0.56 | 4.49E−77 |
| member 1A | | |
| Alpha-1-antichymotrypsin complex | 0.50 | 3.89E−75 |
| Anthrax toxin receptor 2 | −0.45 | 2.93E−73 |
| Transgelin | 0.65 | 8.46E−67 |
| Death within 15 years | | |
| Growth/differentiation factor 15 | 1.03 | 3.38E−154 |
| WAP four-disulfide core domain protein 2 | 0.74 | 3.76E−114 |
| Macrophage metalloelastase | 0.62 | 7.05E−85 |
| Angiopoietin-2 | 0.54 | 6.20E−79 |
| Thrombospondin-2 | 0.46 | 6.69E−69 |
| Retinoblastoma-like protein 2 | 0.48 | 3.58E−67 |
| Anthrax toxin receptor 2 | −0.42 | 3.42E−65 |
| Tumor necrosis factor receptor superfamily | 0.50 | 3.29E−63 |
| member 1A | | |
| Sushi, von Willebrand factor type A, EGF | 0.51 | 1.64E−60 |
| and pentraxin domain-containing protein 1 | | |
| C5a anaphylatoxin | 0.44 | 2.43E−58 |

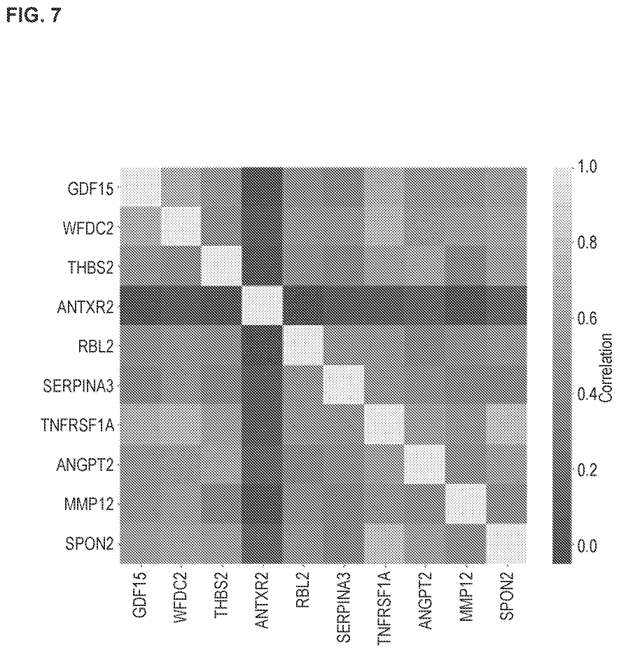

In Table 5, for example, having a greater level of GDF15 protein provides a subject with an increased risk of mortality; and having a greater level of anthrax receptor 2 provides a subject with a decreased risk of mortality. For proteins that are not predictive or are less predictive of mortality, their beta value is 0 or closer to 0. The proteins with the highest associations were rather correlated. For the top ten proteins associating with all-cause mortality within five years, most intercorrelations after correcting for age and sex were 0.4-0.6 (FIG. 7). In contrast, the average correlation between any protein pair of the 4,905 proteins was 0.34.

When univariate associations were examined separately for different causes of death, the top two proteins for all-cause mortality were among the top five proteins for all the death categories except for the nervous system category that had a different protein profile. All top five proteins for neoplasms were also in the top ten for all-cause mortality (Table 6).

TABLE 6
Top single protein associations with different
causes of death within five years.

| Protein | Beta | P-value |
|---|---|---|
| Neoplasms | | |
| Growth/differentiation factor 15 | 1.42 | 3.83E−140 |
| Thrombospondin-2 | 0.77 | 2.02E−90 |
| Retinoblastoma-like protein 2 | 0.85 | 2.29E−88 |
| WAP four-disulfide core domain protein 2 | 0.88 | 6.96E−83 |
| Alpha-1-antichymotrypsin complex | 0.75 | 5.92E−77 |
| Nervous system | | |
| Hematopoietic prostaglandin D synthase | −0.66 | 9.24E−08 |
| Anthrax toxin receptor 2 | −0.60 | 1.41E−07 |
| Disintegrin and metalloproteinase domain- | −0.99 | 5.86E−07 |
| containing protein 22 | | |
| Growth hormone receptor | −0.72 | 9.83E−07 |
| Insulin-like growth factor-binding protein 2 | 0.86 | 2.55E−06 |
| Circulatory system | | |
| Growth/differentiation factor 15 | 1.01 | 1.03E−42 |
| WAP four-disulfide core domain protein 2 | 0.85 | 8.93E−42 |
| N-terminal pro-BNP | 0.66 | 2.73E−34 |
| Transmembrane emp24 domain-containing | 0.53 | 5.62E−32 |
| protein 10 | | |
| Ribonuclease pancreatic | 0.57 | 6.63E−32 |
| Respiratory system | | |
| WAP four-disulfide core domain protein 2 | 1.12 | 2.96E−21 |
| Growth/differentiation factor 15 | 1.12 | 1.32E−16 |
| Troponin T. cardiac muscle | 0.62 | 1.02E−14 |
| Cartilage intermediate layer protein 2 | −0.82 | 4.48E−13 |
| Hematopoietic prostaglandin D synthase | −0.72 | 7.09E−13 |
| Other | | |
| Growth/differentiation factor 15 | 1.13 | 3.05E−22 |
| Tyrosine-protein kinase transmembrane | 0.53 | 6.28E−19 |
| receptor ROR2 | | |
| WAP four-disulfide core domain protein 2 | 0.85 | 1.35E−18 |
| Beta-2-microglobulin | 0.50 | 1.77E−18 |
| Tumor necrosis factor receptor superfamily | 0.66 | 1.22E−17 |
| member 1A | | |

Ranking of Included Proteins

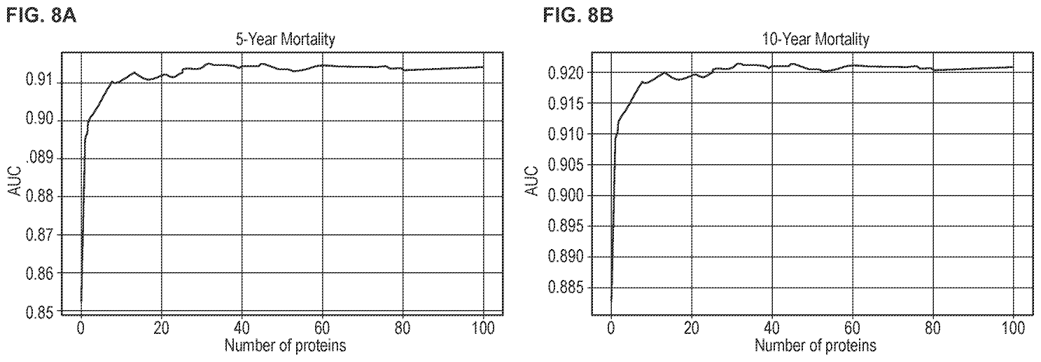

Most of the discrimination performance can be achieved by using only a few proteins in addition to and age and sex. Five, ten, or twenty proteins, selected with a forward selection, yielded AUCs of 0.905, 0.910, and 0.912 for five-year prediction and 0.913, 0.917, and 0.919 for ten-year prediction (FIG. 8A-B, Table 7).

TABLE 7
Order of proteins selected into an all-cause mortality
prediction model on top of age and sex.

| | Forward | L1 | Included |
|---|---|---|---|
| | selection | bootstrap | in 1000 |
| | order | order | bootstraps |
| Death within 5 years | | | |
| Growth/differentiation factor 15 | 1 | 6 | 997 |
| Anthrax toxin receptor 2 | 2 | 81 | 801 |
| Insulin-like growth factor-binding protein 2 | 3 | 161 | 656 |
| Serum amyloid A-1 protein | 4 | 12 | 990 |
| Insulin-like growth factor-binding protein 6 | 5 | 7 | 997 |
| Spondin-1 | 6 | 182 | 624 |
| Tetranectin | 7 | 1 | 1000 |
| dCTP pyrophosphatase 1 | 8 | 2 | 999 |
| Transgelin | 9 | 31 | 945 |
| Tumor necrosis factor receptor superfamily member EDAR | 10 | 49 | 902 |
| Alpha-1-antichymotrypsin complex | 11 | 21 | 976 |
| Erythropoietin | 12 | 5 | 998 |
| Hematopoietic prostaglandin D synthase | 13 | 8 | 995 |
| Macrophage metalloelastase | 14 | 4 | 999 |
| Complement component C7 | 15 | 54 | 874 |
| L-Selectin | 16 | 25 | 969 |
| Netrin receptor UNC5B | 17 | 16 | 985 |
| Brorin | 18 | 34 | 941 |
| WAP four-disulfide core domain protein 2 | 19 | 24 | 970 |
| Serum amyloid A-2 protein | 20 | 167 | 650 |
| Event within 10 years | | | |
| Growth/differentiation factor 15 | 1 | 25 | 980 |
| Anthrax toxin receptor 2 | 2 | 79 | 865 |
| Insulin-like growth factor-binding protein 2 | 3 | 302 | 538 |
| Alpha-1-antichymotrypsin complex | 4 | 19 | 988 |
| Epidermal growth factor receptor | 5 | 14 | 996 |
| Sushi, von Willebrand factor type A, EGF and pentraxin | 6 | 350 | 449 |
| domain-containing protein 1 | | | |
| Tetranectin | 7 | 84 | 857 |
| Spondin-1 | 8 | 229 | 622 |
| Serine protease inhibitor Kazal-type 5 | 9 | 9 | 998 |
| WAP four-disulfide core domain protein 2 | 10 | 5 | 999 |
| Immunoglobulin superfamily member 3 | 11 | 11 | 997 |
| Fc receptor-like protein 1 | 12 | 22 | 985 |
| Pleiotrophin | 13 | 34 | 962 |
| RGM domain family member B | 14 | 17 | 991 |
| Ephrin type-B receptor 2 | 15 | 8 | 999 |
| Transgelin | 16 | 66 | 888 |
| Triggering receptor expressed on myeloid cells 1 | 17 | 77 | 867 |
| Elafin | 18 | 3 | 1000 |
| WNT1-inducible-signaling pathway protein 2 | 19 | 18 | 990 |
| N-terminal pro-BNP | 20 | 24 | 983 |

Associated Phenotypes

The 8,814 participants from the DHS underwent deep phenotyping at the time of sample collection. Although only 0.3% of them died (n=25) during the study period, we calculated for all 8,814, their predicted five-year risk of all-cause mortality using the protein all-cause mortality prediction model and correlated that with ten health and frailty related phenotypes. The predicted risk had a significant negative correlation with the maximum exercise time in an exercise tolerance test, max grip strength, FEV1, number of codes in a digit coding test, and lean appendicular body mass. In contrast, the predicted risk correlated positively with time spent in a trail making test, resting heart rate, and average length from neck to waist over the back. BMI and non-HDL cholesterol levels were not found to be significantly correlated with predicted mortality risk (Table 8).

TABLE 8
Correlation of five-year mortality risk predicted by the protein
model with frailty related phenotypes in the DHS dataset.

| Phenotype | # 18+ | Correlation | P-value | # 60+ | Correlation | P-value |
|---|---|---|---|---|---|---|
| ECG Max exercise time | 6930 | −0.18 | 1.2E−50 | 2334 | −0.25 | 1.4E−34 |
| Max grip strength height | 8737 | −0.10 | 1.1E−21 | 3637 | −0.16 | 1.5E−21 |
| corrected | | | | | | |
| FEV1 | 8015 | −0.15 | 1.3E−40 | 3217 | −0.18 | 1.5E−25 |
| Digit coding: number of codes | 8562 | −0.09 | 6.1E−17 | 3530 | −0.14 | 1.5E−17 |
| minus errors | | | | | | |
| Trail making test B: time | 8485 | 0.09 | 2.7E−16 | 3475 | 0.10 | 1.1E−09 |
| ECG heart rate resting | 6688 | 0.08 | 2.0E−10 | 2851 | 0.08 | 6.0E−05 |
| Bodyscan: Neck average to waist | 8022 | 0.08 | 7.0E−12 | 3300 | 0.12 | 1.0E−11 |
| back adjusted for height | | | | | | |
| DXA Clinical Body Appendicular | 8711 | −0.11 | 2.8E−23 | 3643 | −0.16 | 5.9E−22 |
| Lean/Height² | | | | | | |
| Non-HDL cholesterol, not using | 6397 | −0.01 | 6.2E−01 | 2181 | 0.00 | 9.0E−01 |
| statins | | | | | | |
| Non-HDL cholesterol, using | 1525 | −0.03 | 1.9E−01 | 1181 | −0.04 | 1.7E−01 |
| statins | | | | | | |
| BMI | 8812 | 0.03 | 8.7E−03 | 3683 | 0.01 | 5.5E−01 |

Also, information on various diseases and other traits collected through the Icelandic health system was available for most participants. We looked at how six traits, known to be risk factors for mortality, are associated with the predicted risk in the combined DHS and VSP2 datasets. The protein model predicted higher mortality risk for participants with T2D, MI, CAD, or cancer, and those who smoked, but predicted risk did not associate with clonal hematopoiesis (Table 9).

TABLE 9
Associations of five-year mortality risk predicted by the protein
model with of binary risk-factors in the DHS and VSP2 datasets.

| | 18+ | | | | 60+ | | | |
|---|---|---|---|---|---|---|---|---|
| | | | Difference | | | | Difference | |
| | | | mean risk | P- | | | of mean risk | P- |
| Phenotype | Control | Cases | in sd | value | Control | Cases | in sd | value |
| Diabetes | 14917 | 695 | 0.89 | 2.7E−118 | 5819 | 476 | 0.82 | 1.2E−67 |
| MI | 14969 | 643 | 0.62 | 6.5E−54 | 5002 | 1293 | 0.36 | 1.7E−30 |
| CAD | 14098 | 1514 | 0.40 | 4.7E−50 | 5779 | 516 | 0.48 | 4.9E−26 |
| Cancer | 14401 | 1211 | 0.33 | 2.7E−28 | 5376 | 919 | 0.28 | 9.7E−15 |
| Smoker | 14147 | 1465 | 0.33 | 5.1E−34 | 5750 | 545 | 0.40 | 8.5E−19 |
| Clonal | 7518 | 587 | 0.09 | 2.8E−02 | 3022 | 474 | 0.04 | 3.3E−01 |
| hematopoiesis | | | | | | | | |

Conclusion

By analyzing levels of 4,905 plasma proteins in 22,913 participants, a predictor was developed, built on age, sex, and 81 to 219 proteins that outperforms a predictor composed of traditional risk factors both for long- and short-term prediction. This was true both for participants 18 years or older and when restricting to participants over 60 years of age.

Adding only the plasma protein GDF15 to the age and sex model yielded a model superior to the baseline model that included traditional risk factors. GDF15 has previously been identified as an essential biomarker for all-cause mortality (Ho, J. E. et al. Protein biomarkers of cardiovascular disease and mortality in the community. J. Am. Heart Assoc. 7, (2018); Wiklund, F. E. et al. Macrophage inhibitory cytokine-1 (MIC-1/GDF15): A new marker of all-cause mortality: Serum MIC-1/GDF15 and mortality risk. Aging Cell 9, 1057-1064 (2010)). However, performance increased considerably by adding more proteins to the age and sex model. The best short-term prediction model had fewer proteins than the best long-term prediction model. This may be partly explained by the availability of more cases for longer-term prediction, making it possible to utilize more features without overfitting. The long-term prediction also includes short-term prediction; therefore, both long-term and short-term risk factors have to be considered, making it a more complicated problem.

In a group of 60 to 80 years old, the protein model could identify a group of 5% with an 88% probability of dying within ten years and a 67% probability of dying within five years. Furthermore, the protein model could identify a 5% group with a 1% probability of death within ten years. In contrast, a similar high-risk group identified by the baseline model had a 63% probability of dying within ten years and a 38% probability of dying within five years, while a 5% low-risk group had a 6% probability of dying within ten years.

The protein model predicted mortality within five years more accurately than the baseline model for all the different causes of death analyzed, i.e., neoplasms, the nervous system, the circulatory system, the respiratory system, and other reasons. Similarly, the model predicted those who did not die within five years at a lower risk. This suggests that the protein model is predicting all-cause mortality rather than being biased to a specific cause of death. Of the different causes of death, the poorest prediction by all models was for cancer death.

The predicted risk by the protein model correlates well, in an independent dataset, with phenotypes that can be considered as measures of health and frailty (Lara, J. et al. Towards measurement of the Healthy Ageing Phenotype in lifestyle-based intervention studies. Maturitas 76, 189-199 (2013)). Participants at higher predicted mortality risk had lower endurance in an exercise test, weaker grip, lower FEV1, performed worse on a digit coding test, took longer time at a trail making test, had faster-resting heart rate, and had less appendicular lean body mass. Interestingly, the higher predicted risk was also correlated with greater length from neck to waist over the back, which could be due to age-related kyphosis (Nishiwaki, Y. et al. Association of thoracic kyphosis with subjective poor health, functional activity and blood pressure in the community-dwelling elderly. Environ. Health Prev. Med. 12, 246-250 (2007)).

The study's main limitation is that some of the risk factors were not available at the time of plasma collection for some participants. Therefore some imputation had to be employed for medication data, BMI, and smoking. Furthermore, ApoB levels were used as a surrogate for non-HDL cholesterol and hypertension medication for blood pressure. Risk factors, such as levels of creatinine, platelets, and triglycerides, were not included in the baseline model. The baseline model would also have benefited from the inclusion of other diseases than T2D, CAD, previous MI, and previous cancer diagnosis. The types of cancer, diagnosis times, and all medication information would probably also have improved the baseline model. A possible limitation is also that the training and testing data are enriched with cancer patients. Therefore it is not a random sample of the population. However, reassuringly, the model could predict other causes of death even better than that of cancer.

The most significant advantage of the protein approach is that it only needs a single blood draw to get prediction accuracy better than a model that includes multiple risk factor measurements and disease diagnosis. The recent technical advantages in simultaneously measuring a large number of proteins open up the possibility of evaluating, accurately, an individual's state of health from one blood draw. If the number of proteins is a limitation, only measuring 1-20 proteins still yields a powerful predictor.

A good all-cause mortality predictor could be useful as a clinical study endpoint to determine treatment effects, making it possible to get some results without waiting for participants to die. Similarly, it could also be useful to help assess disease treatments. Since the proteins used in this study were not normalized on the sample set, this predictor could be used directly on individual protein measurements from the SOMAscan platform.

Death is the final event and can never be considered trivial. Any further insights into the long-term causes of death will always be valuable. This study shows the power of protein levels in plasma as predictors of death.

### Example 3

The Effect of Repatha® (Evolocumab) on Decreasing Risk of Mortality

Subjects (e.g., human patients) determined to have an elevated level of LDL bad cholesterol are chosen for a study to determine the effects of Repatha® (evolocumab) on decreasing the risk of mortality in the subject.

Prior to treatment with Repatha®, protein levels of at least one of growth/differentiation factor 15 (GDF15); thrombospondin-2 (TSP-2); retinoblastoma-like protein 2 (RBL2); WAP four-disulfide core domain protein 2 (WFDC2); anthrax toxin receptor 2 (ANTRX2); alpha-1-antichymotrypsin complex (ACT COMPLEX or SERPINA3); spondin-1 (SPON1); spondin-2 (SPON2); serum amyloid A-1 protein (SAA1); serum amyloid A-2 protein (SAA2); C5a anaphylatoxin (C5aAT); tumor necrosis factor receptor superfamily member 1A (TNFRSF1A); angiopoietin-2 (ANG2); macrophage metalloelastase (MMP12); transgelin (TAGLN); sushi, von Willebrand factor type A, EGF and pentraxin domain-containing protein 1 (SVEP1); hematopoietic prostaglandin D synthase (HPGDS); disintegrin and metalloproteinase domain-containing protein 22 (ADAM22); growth hormone receptor (GHR); insulin-like growth factor-binding protein 2 (IGFPB2); N-terminal pro-BNP (NTproBNP); transmembrane emp24 domain-containing protein 10 (TMED10); pancreatic ribonuclease (RNase1); cardiac troponin T (cTnT); cartilage intermediate layer protein 2 (CILP2); tyrosine-protein kinase transmembrane receptor ROR2 (ROR2); beta-2-microglobulin (beta-2-M); insulin-like growth factor-binding protein 6 (IGFPB6); tetranectin (TN); dCTP pyrophosphatase 1 (DCTPP1); tumor necrosis factor receptor superfamily member EDAR (EDAR); erythropoietin (EPO); complement component C7 (C7); L-Selectin (CD62L); netrin receptor UNC5B (UNCB5); brorin; epidermal growth factor receptor (EGFR); serine protease inhibitor Kazal-type 5 (SPINK5); immunoglobulin superfamily member 3 (IGSF3); Fc receptor-like protein 1 (FCRL1); pleiotrophin (PTN); RGM domain family member B (RGMB); ephrin type-B receptor 2 (EPHB2); triggering receptor expressed on myeloid cells 1 (TREM-1); elafin; WNT1-inducible-signaling pathway protein 2 (WISP-2); or a combination comprising any two or more of these proteins, or a combination comprising all of these proteins are measured in a sample of blood plasma from the subject.

Repatha® is administered subcutaneously: 140 mg every 2 weeks or 420 mg once monthly in abdomen, thigh, or upper arm. The 420 mg dose of Repatha® is administered over 9 minutes by using the single-use on-body infusor with prefilled cartridge, or by giving 3 injections consecutively within 30 minutes using the single-use prefilled autoinjector or single-use prefilled syringe.

After treatment with Repatha®, protein levels are measured at 4 weeks, 8 weeks, 12 weeks, 24 weeks, 48 weeks, and 72 weeks.

Decreases in the level of one or more of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 are shown in the subject after treatment compared to the level of one or more of these proteins in the subject prior to treatment, indicating that Repatha® (evolocumab) decreases the risk of mortality in the subject.

Increases in the level of one or more of ANTRX2, HPGDS, ADAM22, GHR, or CILP2 are shown in the subject after treatment compared to the level of one or more of these proteins in the subject prior to treatment, indicating that Repatha® (evolocumab) decreases the risk of mortality in the subject.

### Example 4

The Effect of Enbrel® (etanercept) on Decreasing Risk of Mortality

Enbrel® is a tumor necrosis factor (TNF) blocker indicated for the treatment of: rheumatoid arthritis (RA), polyarticular juvenile idiopathic arthritis (JIA) in patients aged 2 years or older, psoriatic arthritis, ankylosing spondylitis (AS), and plaque psoriasis (PsO) in patients 4 years or older.

Subjects (e.g., human patients) determined to have RA are chosen for a study to determine the effects of Enbrel® (etanercept) on decreasing the risk of mortality in the subject.

Prior to treatment with Enbrel®, protein levels of at least one of growth/differentiation factor 15 (GDF15); thrombospondin-2 (TSP-2); retinoblastoma-like protein 2 (RBL2); WAP four-disulfide core domain protein 2 (WFDC2); anthrax toxin receptor 2 (ANTRX2); alpha-1-antichymotrypsin complex (ACT COMPLEX or SERPINA3); spondin-1 (SPON1); spondin-2 (SPON2); serum amyloid A-1 protein (SAA1); serum amyloid A-2 protein (SAA2); C5a anaphylatoxin (C5aAT); tumor necrosis factor receptor superfamily member 1A (TNFRSF1A); angiopoietin-2 (ANG2); macrophage metalloelastase (MMP12); transgelin (TAGLN); sushi, von Willebrand factor type A, EGF and pentraxin domain-containing protein 1 (SVEP1); hematopoietic prostaglandin D synthase (HPGDS); disintegrin and metalloproteinase domain-containing protein 22 (ADAM22); growth hormone receptor (GHR); insulin-like growth factor-binding protein 2 (IGFPB2); N-terminal pro-BNP (NTproBNP); transmembrane emp24 domain-containing protein 10 (TMED10); pancreatic ribonuclease (RNase1); cardiac troponin T (cTnT); cartilage intermediate layer protein 2 (CILP2); tyrosine-protein kinase transmembrane receptor ROR2 (ROR2); beta-2-microglobulin (beta-2-M); insulin-like growth factor-binding protein 6 (IGFPB6); tetranectin (TN); dCTP pyrophosphatase 1 (DCTPP1); tumor necrosis factor receptor superfamily member EDAR (EDAR); erythropoietin (EPO); complement component C7 (C7); L-Selectin (CD62L); netrin receptor UNC5B (UNCB5); brorin; epidermal growth factor receptor (EGFR); serine protease inhibitor Kazal-type 5 (SPINK5); immunoglobulin superfamily member 3 (IGSF3); Fc receptor-like protein 1 (FCRL1); pleiotrophin (PTN); RGM domain family member B (RGMB); ephrin type-B receptor 2 (EPHB2); triggering receptor expressed on myeloid cells 1 (TREM-1); elafin; WNT1-inducible-signaling pathway protein 2 (WISP-2); or a combination comprising any two or more of these proteins, or a combination comprising all of these proteins are measured in a sample of blood plasma from the subject.

Enbrel® is administered subcutaneously: 50 mg once weekly with or without methotrexate (MTX). After treatment with Enbrel®, protein levels are measured at 4 weeks, 8 weeks, 12 weeks, 24 weeks, 48 weeks, and 72 weeks.

Decreases in the level of one or more of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 are shown in the subject after treatment compared to the level of one or more of these proteins in the subject prior to treatment, indicating that Enbrel® decreases the risk of mortality in the subject.

Increases in the level of one or more of ANTRX2, HPGDS, ADAM22, GHR, or CILP2 are shown in the subject after treatment compared to the level of one or more of these proteins in the subject prior to treatment, indicating that Enbrel® decreases the risk of mortality in the subject.

### Example 5

The Effect of Tezepelumab on Decreasing Risk of Mortality

Tezepelumab is a human monoclonal antibody designed for the treatment of asthma and atopic dermatitis. It blocks thymic stromal lymphopoietin, an epithelial cytokine that has been suggested to be critical in the initiation and persistence of airway inflammation.

Subjects (e.g., human patients) determined to have asthma are chosen for a study to determine the effects of tezepelumab on decreasing the risk of mortality in the subject.

Prior to treatment with Enbrel®, protein levels of at least one of growth/differentiation factor 15 (GDF15); thrombospondin-2 (TSP-2); retinoblastoma-like protein 2 (RBL2); WAP four-disulfide core domain protein 2 (WFDC2); anthrax toxin receptor 2 (ANTRX2); alpha-1-antichymotrypsin complex (ACT COMPLEX or SERPINA3); spondin-1 (SPON1); spondin-2 (SPON2); serum amyloid A-1 protein (SAA1); serum amyloid A-2 protein (SAA2); C5a anaphylatoxin (C5aAT); tumor necrosis factor receptor superfamily member 1A (TNFRSF1A); angiopoietin-2 (ANG2); macrophage metalloelastase (MMP12); transgelin (TAGLN); sushi, von Willebrand factor type A, EGF and pentraxin domain-containing protein 1 (SVEP1); hematopoietic prostaglandin D synthase (HPGDS); disintegrin and metalloproteinase domain-containing protein 22 (ADAM22); growth hormone receptor (GHR); insulin-like growth factor-binding protein 2 (IGFPB2); N-terminal pro-BNP (NTproBNP); transmembrane emp24 domain-containing protein 10 (TMED10); pancreatic ribonuclease (RNase1); cardiac troponin T (cTnT); cartilage intermediate layer protein 2 (CILP2); tyrosine-protein kinase transmembrane receptor ROR2 (ROR2); beta-2-microglobulin (beta-2-M); insulin-like growth factor-binding protein 6 (IGFPB6); tetranectin (TN); dCTP pyrophosphatase 1 (DCTPP1); tumor necrosis factor receptor superfamily member EDAR (EDAR); erythropoietin (EPO); complement component C7 (C7); L-Selectin (CD62L); netrin receptor UNC5B (UNCB5); brorin; epidermal growth factor receptor (EGFR); serine protease inhibitor Kazal-type 5 (SPINK5); immunoglobulin superfamily member 3 (IGSF3); Fc receptor-like protein 1 (FCRL1); pleiotrophin (PTN); RGM domain family member B (RGMB); ephrin type-B receptor 2 (EPHB2); triggering receptor expressed on myeloid cells 1 (TREM-1); elafin; WNT1-inducible-signaling pathway protein 2 (WISP-2); or a combination comprising any two or more of these proteins, or a combination comprising all of these proteins are measured in a sample of blood plasma from the subject.

During the treatment period, one dose of 210 mg tezepelumab is administered via a single-use accessorized pre-filled syringes (APFS) or Al subcutaneously (SC) every 4 weeks (Q4W) starting at Visit 2 (Week 0) until Visit 7 (Week 20). Subjects are administered tezepelumab at the site during Visits 2 (Week 0), 3 (Week 4), 4 (Week 8) and 7 (Week 20). At-home administration of tezepelumab occurs during Visit 5 (Week 12) and Visit 6 (Week 16). After treatment with tezepelumab, protein levels are measured at 4 weeks, 8 weeks, 12 weeks, 24 weeks, 48 weeks, and 72 weeks.

Decreases in the level of one or more of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 are shown in the subject after treatment compared to the level of one or more of these proteins in the subject prior to treatment, indicating that tezepelumab decreases the risk of mortality in the subject.

Increases in the level of one or more of ANTRX2, HPGDS, ADAM22, GHR, or CILP2 are shown in the subject after treatment compared to the level of one or more of these proteins in the subject prior to treatment, indicating that tezepelumab decreases the risk of mortality in the subject.

### Example 6

The Effect of Prolia® or XGEVA® (denosumab) on Decreasing Risk of Mortality

Prolia® (denosumab) is a RANK ligand (RANKL) inhibitor indicated for treatment of postmenopausal women with osteoporosis at high risk for fracture, treatment to increase bone mass in men with osteoporosis at high risk for fracture, treatment of glucocorticoid-induced osteoporosis in men and women at high risk for fracture, treatment to increase bone mass in men at high risk for fracture receiving androgen deprivation therapy for nonmetastatic prostate cancer, and treatment to increase bone mass in women at high risk for fracture receiving adjuvant aromatase inhibitor therapy for breast cancer.

Subjects (e.g., human patients) determined to be at high risk for fracture for various conditions, as set out herein above, are chosen for a study to determine the effects of denosumab on decreasing the risk of mortality in the subject.

Prior to treatment with denosumab, protein levels of at least one of growth/differentiation factor 15 (GDF15); thrombospondin-2 (TSP-2); retinoblastoma-like protein 2 (RBL2); WAP four-disulfide core domain protein 2 (WFDC2); anthrax toxin receptor 2 (ANTRX2); alpha-1-antichymotrypsin complex (ACT COMPLEX or SERPINA3); spondin-1 (SPON1); spondin-2 (SPON2); serum amyloid A-1 protein (SAA1); serum amyloid A-2 protein (SAA2); C5a anaphylatoxin (C5aAT); tumor necrosis factor receptor superfamily member 1A (TNFRSF1A); angiopoietin-2 (ANG2); macrophage metalloelastase (MMP12); transgelin (TAGLN); sushi, von Willebrand factor type A, EGF and pentraxin domain-containing protein 1 (SVEP1); hematopoietic prostaglandin D synthase (HPGDS); disintegrin and metalloproteinase domain-containing protein 22 (ADAM22); growth hormone receptor (GHR); insulin-like growth factor-binding protein 2 (IGFPB2); N-terminal pro-BNP (NTproBNP); transmembrane emp24 domain-containing protein 10 (TMED10); pancreatic ribonuclease (RNase1); cardiac troponin T (cTnT); cartilage intermediate layer protein 2 (CILP2); tyrosine-protein kinase transmembrane receptor ROR2 (ROR2); beta-2-microglobulin (beta-2-M); insulin-like growth factor-binding protein 6 (IGFPB6); tetranectin (TN); dCTP pyrophosphatase 1 (DCTPP1); tumor necrosis factor receptor superfamily member EDAR (EDAR); erythropoietin (EPO); complement component C7 (C7); L-Selectin (CD62L); netrin receptor UNC5B (UNCB5); brorin; epidermal growth factor receptor (EGFR); serine protease inhibitor Kazal-type 5 (SPINK5); immunoglobulin superfamily member 3 (IGSF3); Fc receptor-like protein 1 (FCRL1); pleiotrophin (PTN); RGM domain family member B (RGMB); ephrin type-B receptor 2 (EPHB2); triggering receptor expressed on myeloid cells 1 (TREM-1); elafin; WNT1-inducible-signaling pathway protein 2 (WISP-2); or a combination comprising any two or more of these proteins, or a combination comprising all of these proteins are measured in a sample of blood plasma from the subject.

During the treatment period, one dose of 60 mg in a 1 mL solution denosumab is administered via a single-dose prefilled syringe. Denosumab is administered 60 mg every 6 months as a subcutaneous injection in the upper arm, upper thigh, or abdomen. After treatment with denosumab, protein levels are measured at 4 weeks, 8 weeks, 12 weeks, 24 weeks, 48 weeks, and 72 weeks.

Decreases in the level of one or more of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 are shown in the subject after treatment compared to the level of one or more of these proteins in the subject prior to treatment, indicating that denosumab decreases the risk of mortality in the subject.

Increases in the level of one or more of ANTRX2, HPGDS, ADAM22, GHR, or CILP2 are shown in the subject after treatment compared to the level of one or more of these proteins in the subject prior to treatment, indicating that denosumab decreases the risk of mortality in the subject.

The disclosure has been described in terms of particular embodiments found or proposed to comprise specific modes for the practice of the disclosure. Various modifications and variations of the disclosure will be apparent to those skilled in the art without departing from the scope and spirit of the disclosure. Although the disclosure has been described in connection with specific embodiments, it should be understood that the methods of the disclosure as claimed should not be unduly limited to such specific embodiments. Indeed, various modifications of the described modes for carrying out the methods that are obvious to those skilled in the relevant fields are intended to be within the scope of the following claims.

What is claimed is:

1. A method of treating a subject to reduce risk of mortality of the subject, the method comprising:
measuring the level of a protein in a biological sample from the subject;
comparing the measured level of the protein to a reference level for the protein, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2); and
administering to the subject an effective dose of a therapeutic treatment to decrease the risk of mortality of the subject if
(i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level and/or;
(ii) the level of ANTRX2, HPGDS, ADAM22, GHR, or CILP2 is decreased relative to the reference level.

2. A method for determining the efficacy of a therapeutic treatment in a subject to reduce risk of mortality of a subject, the method comprising:
measuring the level of a protein in a biological sample from the subject before the therapeutic treatment, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2);
measuring the level of the protein in a biological sample from the subject after administering the treatment; and
determining that the treatment is effective in reducing risk of mortality of the subject if
(i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is decreased relative to the reference level or to the level in the sample from the subject before treatment; and/or
(ii) the level of ANTRX2, HPGDS, ADAM22, GHR, or CILP2 is increased relative to the reference level or to the level in the sample from the subject before treatment.

3. The method of claim 1 or 2, comprising measuring the level of each of any two or more of the proteins of (a)-(at); and comparing the measured level of each of the proteins to the reference level for each of the proteins.

4. The method of any one of claims 1-3, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) anthrax toxin receptor 2 (ANTRX2).

5. The method of any one of claims 1-3, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-2 (SPON2);
(h) serum amyloid A-1 protein (SAA1);
(i) serum amyloid A-2 protein (SAA2); and/or
(j) C5a anaphylatoxin (C5aAT).

6. The method of any one of claims 1-3, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSFiA);
(h) angiopoietin-2 (ANG2);
(i) macrophage metalloelastase (MMP12); and/or
(j) spondin-2 (SPON2).

7. The method of any one of claims 1-3, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSF1A);
(h) angiopoietin-2 (ANG2);
(i) macrophage metalloelastase (MMP12); and/or
(j) transgelin (TAGLN).

8. The method of any one of claims 1-3, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) angiopoietin-2 (ANG2);
(g) tumor necrosis factor receptor superfamily member 1A (TNFRSF1A);
(h) macrophage metalloelastase (MMP12);
(i) sushi, von Willebrand factor type A, EGF and pentraxin domain-containing protein 1 (SVEP1); or
(j) C5a anaphylatoxin (C5aAT).

9. The method of any one of claims 1-3, wherein the protein and/or proteins is
(a) growth/differentiation factor 15 (GDF15);
(b) thrombospondin-2;
(c) retinoblastoma-like protein 2;
(d) WAP four-disulfide core domain protein 2;
(e) anthrax toxin receptor 2;
(f) alpha-1-antichymotrypsin complex;
(g) spondin-2;
(h) serum amyloid A-1;
(i) serum amyloid A-2;
(j) C5a anaphylatoxin;
(k) tumor necrosis factor receptor superfamily member 1A;
(l) angiopoietin-2;
(m) macrophage metalloelastase;
(n) transgelin; and/or
(o) sushi, von Willebrand factor type A, EGF and pentraxin domain-containing protein 1.

10. The method of any one of claims 1-3, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) alpha-1-antichymotrypsin complex (ACT COMPLEX or SERPINA3).

11. The method of any one of claims 1-3, wherein the protein and/or proteins is
(a) hematopoietic prostaglandin D synthase (HPGDS);
(b) anthrax toxin receptor 2 (ANTRX2);
(c) disintegrin and metalloproteinase domain-containing protein 22 (ADAM22);
(d) growth hormone receptor (GHR); and/or
(e) insulin-like growth factor-binding protein 2 (IGFPB2).

12. The method of any one of claims 1-3, wherein the protein and/or proteins is
(a) growth/differentiation factor 15 (GDF15);
(b) WAP four-disulfide core domain protein 2 (WFDC2);
(c) cardiac troponin T (cTnT);
(d) cartilage intermediate layer protein 2 (CILP2); and/or
(e) hematopoietic prostaglandin D synthase (HPGDS).

13. The method of any one of claims 1-3, wherein the protein and/or proteins is
(a) growth/differentiation factor 15 (GDF15);
(b) WAP four-disulfide core domain protein 2 (WFDC2);
(c)N-terminal pro-BNP (NTproBNP);
(d) transmembrane emp24 domain-containing protein 10 (TMED10); and/or
(e) pancreatic ribonuclease (RNase1).

14. The method of any one of claims 1-3, wherein the protein and/or proteins is
(a) growth/differentiation factor 15 (GDF15);
(b) tyrosine-protein kinase transmembrane receptor ROR2 (ROR2);
(c) WAP four-disulfide core domain protein 2 (WFDC2);
(d) beta-2-microglobulin (beta-2-M); and/or
(e) tumor necrosis factor receptor superfamily member 1A (TNFRSF1A).

15. The method of any one of claims 1-3, wherein the therapeutic treatment is any one or more of Aimovig™ (erenumab-aooe; AMG 334), Aranesp® (darbepoetin alfa), AVSOLA™ (infliximab-axxq), BLINCYTO® (blinatumomab), Corlanor® (ivabradine), Enbrel® (etanercept), EPOGEN® (epoetin alfa), EVENITY™ (romosozumab-aqqg), IMLYGIC® (talimogene laherparepvec), Kanjinti™ (trastuzumab-anns), Kyprolis® (carfilzomib), MVASI™ (bevacizumab-awwb), Neulasta® (pegfilgrastim), NEUPOGEN® (filgrastim), Nplate® (romiplostim), Otezla® (apremilast), Parsabiv™ (etelcalcetide), Prolia® (denosumab), Repatha® (evolocumab), Sensipar® (cinacalcet), Vectibix® (panitumumab), or XGEVA® (denosumab).

16. The method of any one of claims 1-3, wherein the therapeutic treatment is a biological or a pharmaceutical drug that
(a) reduces the risk of mortality from a neoplastic disease or disorder;
(b) reduces the risk of mortality from a nervous disease or disorder;
(c) reduces the risk of mortality from a circulatory disease or disorder;
(d) reduces the risk of mortality from a respiratory disease or disorder;
(e) reduces the risk of mortality from inflammation or an inflammatory disease or disorder; and/or
(d) reduces the risk of mortality from an autoimmune disease or disorder.

17. The method of claim 10 or 16, wherein the biological or pharmaceutical drug reduces the risk of mortality from a neoplastic disease or disorder.

18. The method of claim 11 or 16, wherein the biological or pharmaceutical drug reduces the risk of mortality from a nervous disease or disorder.

19. The method of claim 12 or 16, wherein the biological or pharmaceutical drug reduces the risk of mortality from a circulatory disease or disorder.

20. The method of claim 13 or 16, wherein the biological or pharmaceutical drug reduces the risk of mortality from a respiratory disease or disorder.

21. The method of claim 16, wherein the biological or pharmaceutical drug reduces the risk of mortality from an inflammatory disease or disorder.

22. The method of claim 16, wherein the biological or pharmaceutical drug reduces the risk of mortality from an autoimmune disease or disorder.

23. The method of claim 19, wherein the circulatory disease or disorder is hypercholesterolemia, myocardial infarction, and/or stroke.

24. The method of claim 19 or 23, wherein the biological or pharmaceutical drug is evolocumab, atorvastatin, fluvastatin, lovastatin, pitavastatin, pravastatin, rosuvastatin calcium, or simvastatin.

25. The method of claim 24, wherein the biological drug or pharmaceutical drug is evolocumab.

26. The method of claim 20, wherein the respiratory disease or disorder is asthma, allergy, carbon monoxide poisoning, smoke inhalation, chronic bronchitis, emphysema, asbestos poisoning, bronchitis, pulmonary fibrosis, cystic fibrosis/bronchiectasis, lung cancer, embolism, Chronic Obstructive Pulmonary Disease (COPD), adult respiratory distress syndrome, pulmonary hypertension, Celiac's disease, pneumonitis, pneumonia, or pleural effusion.

27. The method of claim 26, wherein the respiratory disease or disorder is asthma.

28. The method of claim 20, 26, or 27, wherein the biological or pharmaceutical drug is tezepelumab, omalizumab, benzalizumab, mepolizumab, or reslizumab.

29. The method of claim 28, wherein the biological or pharmaceutical drug is tezepelumab.

30. The method of claim 21, wherein the inflammatory disease or disorder is rheumatoid arthritis, psoriatic arthritis, plaque psoriasis, or ankylosing spondylitis.

31. The method of claim 21 or 30, wherein the biological or pharmaceutical drug is etanercept, adalimumab, dupilumab, ustekinumab, infliximab, golimumab, or certolizumab pegol.

32. The method of claim 21, 30, or 31, wherein the biological or pharmaceutical drug is etanercept.

33. The method of any of the preceding claims, wherein the reference level is a mean level of the biomarker in a biological sample from a population of subjects determined to be healthy or not to be at an increased risk of mortality.

34. The method of any one of the preceding claims, the method comprising measuring a level of at least two, at least three, at least four, at least five, at least six, at least seven, at least eight, at least nine, at least 10, at least 11, at least 12, at least 13, at least 14, at least 15, at least 16, at least 17, at least 18, at least 19, or at least 20 proteins in the biological sample from the subject.

35. The method of any one of the preceding claims, wherein the biological sample is a blood sample.

36. The method of claim 35, wherein the blood sample is plasma.

37. A method for predicting increased risk of mortality of a subject, the method comprising:
measuring the level of a protein in a biological sample from the subject and comparing the level to a reference level, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSF1A);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2),
wherein the subject is determined to have an increased risk of mortality when
(i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level; and/or
(ii) the level of ANTRX2, HPGDS, ADAM22, GHR, or CILP2 is decreased relative to the reference level.

38. The method of claim 37, comprising measuring the level of each of any two or more of the proteins of (a)-(at); and
comparing the measured level of each of the proteins to the reference level for each of the proteins, wherein the subject is determined to have an increased risk of mortality when
(i) the level of GDF15, TSP-2, RBL2, WFDC2, SERPINA3, SPON1, SPON2, SAA1, SAA2, C5Aat, TNFRSF1A, ANG2, MMP12, TAGLN, SVEP1, IGFPB2, NTproBNP, TMED10, RNase1, cTnT, ROR2, beta-2-M, IGFPB6, tetranectin, DCTPP1, EDAR, EPO, C7, CD62L, UNC5B, brorin, EGFR, SPINK5, IGSF3, FCRL1, PTN, RGMB, EPHB2, TREM-1, elafin, or WISP-2 is increased relative to the reference level; and/or
(ii) the level of ANTRX2, HPGDS, ADAM22, GHR, or CILP2 is decreased relative to the reference level.

39. The method of claim 37 or 38, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) anthrax toxin receptor 2 (ANTRX2).

40. The method of claim 37 or 38, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-2 (SPON2);
(h) serum amyloid A-1 protein (SAA1);
(i) serum amyloid A-2 protein (SAA2); and/or
(j) C5a anaphylatoxin (C5aAT).

41. The method of claim 37 or 38, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSFiA);
(h) angiopoietin-2 (ANG2);
(i) macrophage metalloelastase (MMP12); and/or
(j) spondin-2 (SPON2).

42. The method of claim 37 or 38, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) tumor necrosis factor receptor superfamily member 1A (TNFRSF1A);
(h) angiopoietin-2 (ANG2);
(i) macrophage metalloelastase (MMP12); and/or
(j) transgelin (TAGLN).

43. The method of claim 37 or 38, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) angiopoietin-2 (ANG2);
(g) tumor necrosis factor receptor superfamily member 1A (TNFRSF1A);
(h) macrophage metalloelastase (MMP12);
(i) sushi, von Willebrand factor type A, EGF and pentraxin domain-containing protein 1 (SVEP1); and/or
(j) C5a anaphylatoxin (C5aAT).

44. The method of claim 37 or 38, wherein the protein and/or proteins is
(a) growth/differentiation factor 15 (GDF15);
(b) thrombospondin-2;
(c) retinoblastoma-like protein 2;
(d) WAP four-disulfide core domain protein 2;
(e) anthrax toxin receptor 2;
(f) alpha-1-antichymotrypsin complex;
(g) spondin-2;
(h) serum amyloid A-1;
(i) serum amyloid A-2;
(j) C5a anaphylatoxin;
(k) tumor necrosis factor receptor superfamily member 1A;
(l) angiopoietin-2;
(m) macrophage metalloelastase;
(n) transgelin; and/or
(o) sushi, von Willebrand factor type A, EGF and pentraxin domain-containing protein 1.

45. The method of claim 37 or 38, wherein the protein and/or proteins is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2); and/or
(e) alpha-1-antichymotrypsin complex (ACT COMPLEX or SERPINA3).

46. The method of claim 37 or 38, wherein the protein and/or proteins is
(a) hematopoietic prostaglandin D synthase (HPGDS);
(b) anthrax toxin receptor 2 (ANTRX2);
(c) disintegrin and metalloproteinase domain-containing protein 22 (ADAM22);
(d) growth hormone receptor (GHR); and/or
(e) insulin-like growth factor-binding protein 2 (IGFPB2).

47. The method of claim 37 or 38, wherein the protein and/or proteins is
(a) growth/differentiation factor 15 (GDF15);
(b) WAP four-disulfide core domain protein 2 (WFDC2);
(c) cardiac troponin T (cTnT);
(d) cartilage intermediate layer protein 2 (CILP2); and/or
(e) hematopoietic prostaglandin D synthase (HPGDS).

48. The method of claim 37 or 38, wherein the protein and/or proteins is
(a) growth/differentiation factor 15 (GDF15);
(b) WAP four-disulfide core domain protein 2 (WFDC2);
(c)N-terminal pro-BNP (NTproBNP);
(d) transmembrane emp24 domain-containing protein 10 (TMED10); and/or
(e) pancreatic ribonuclease (RNase1).

49. The method of claim 37 or 38, wherein the protein and/or proteins is
(a) growth/differentiation factor 15 (GDF15);
(b) tyrosine-protein kinase transmembrane receptor ROR2 (ROR2);
(c) WAP four-disulfide core domain protein 2 (WFDC2);
(d) beta-2-microglobulin (beta-2-M); and/or
(e) tumor necrosis factor receptor superfamily member 1A (TNFRSF1A).

50. The method of any one of claims 37, 38, 39 and 43, wherein the subject has an increased risk of mortality within 2 years.

51. The method of any one of claims 37, 38, 40 and 43-48, wherein the subject has an increased risk of mortality within 5 years.

52. The method of any one of claims 37, 38, 41, and 43, wherein the subject has an increased risk of mortality within 10 years.

53. The method of any one of claims 37, 38, 42, and 43, wherein the subject has an increased risk of mortality within 15 years.

54. The method of claim 45, wherein the subject has an increased risk of mortality from a neoplastic disease or disorder.

55. The method of claim 46, wherein the subject has an increased risk of mortality from a nervous system disease or disorder.

56. The method of claim 47, wherein the subject has an increased risk of mortality from a circulatory system disease or disorder.

57. The method of claim 48, wherein the subject has an increased risk of mortality from a respiratory system disease or disorder.

58. The method of any one of claims 37-56, wherein the reference level is a mean level of the biomarker in a biological sample from a population of subjects determined to be healthy or not to be at an increased risk of mortality.

59. The method of any one of claims 37-58, the method comprising measuring the level of at least two, at least three, at least four, at least five, at least six, at least seven, at least eight, at least nine, at least 10, at least 11, at least 12, at least 13, at least 14, at least 15, at least 16, at least 17, at least 18, at least 19, or at least 20 proteins in the biological sample from the subject.

60. The method of any one of claims 37-59, wherein the biological sample is a blood sample.

61. The method of claim 60, wherein the blood sample is plasma.

62. The method of any one of the preceding claims, wherein the mortality is all-cause mortality.

63. The method of any one of claims 37-62, wherein when the subject is predicted to have an increased risk of mortality, the method further comprises administering a therapeutic treatment to the subject to reduce the increased risk.

64. The method of claim 63, wherein the therapeutic treatment is any one or more of Aimovig™ (erenumab-aooe; AMG 334), Aranesp® (darbepoetin alfa), AVSOLA™ (infliximab-axxq), BLINCYTO® (blinatumomab), Corlanor® (ivabradine), Enbrel® (etanercept), EPOGEN® (epoetin alfa), EVENITY™ (romosozumab-aqqg), IMLYGIC® (talimogene laherparepvec), Kanjinti™ (trastuzumab-anns), Kyprolis® (carfilzomib), MVASI™ (bevacizumab-awwb), Neulasta® (pegfilgrastim), NEUPOGEN® (filgrastim), Nplate® (romiplostim), Otezla® (apremilast), Parsabiv™ (etelcalcetide), Prolia® (denosumab), Repatha® (evolocumab), Sensipar® (cinacalcet), Vectibix® (panitumumab), or XGEVA® (denosumab).

65. The method of claim 63 or 64, wherein the therapeutic treatment is a biological or a pharmaceutical drug that
(a) reduces the risk of mortality from a neoplastic disease or disorder;
(b) reduces the risk of mortality from a nervous disease or disorder;
(c) reduces the risk of mortality from a circulatory disease or disorder;
(d) reduces the risk of mortality from a respiratory disease or disorder;
(e) reduces the risk of mortality from inflammation or an inflammatory disease or disorder; and/or
(f) reduces the risk of mortality from an autoimmune disease or disorder.

66. The method of claim 45 or 65, wherein the biological or pharmaceutical drug reduces the risk of mortality from a neoplastic disease or disorder.

67. The method of claim 46 or 65, wherein the biological or pharmaceutical drug reduces the risk of mortality from a nervous disease or disorder.

68. The method of claim 47 or 65, wherein the biological or pharmaceutical drug reduces the risk of mortality from a circulatory disease or disorder.

69. The method of claim 48 or 65, wherein the biological or pharmaceutical drug reduces the risk of mortality from a respiratory disease or disorder.

70. The method of claim 65, wherein the biological or pharmaceutical drug reduces the risk of mortality from an inflammatory disease or disorder.

71. The method of claim 65, wherein the biological or pharmaceutical drug reduces the risk of mortality from an autoimmune disease or disorder.

72. The method of claim 68, wherein the circulatory disease or disorder is hypercholesterolemia, myocardial infarction, and/or stroke.

73. The method of claim 68 or 72, wherein the biological or pharmaceutical drug is evolocumab, atorvastatin, fluvastatin, lovastatin, pitavastatin, pravastatin, rosuvastatin calcium, or simvastatin.

74. The method of claim 73, wherein the biological is evolocumab.

75. The method of claim 69, wherein the respiratory disease or disorder is asthma, allergy, carbon monoxide poisoning, smoke inhalation, chronic bronchitis, emphysema, asbestos poisoning, bronchitis, pulmonary fibrosis, cystic fibrosis/bronchiectasis, lung cancer, embolism, Chronic Obstructive Pulmonary Disease (COPD), adult respiratory distress syndrome, pulmonary hypertension, Celiac's disease, pneumonitis, pneumonia, or pleural effusion.

76. The method of claim 75, wherein the respiratory disease or disorder is asthma.

77. The method of claim 69, 75, or 76, wherein the biological or pharmaceutical drug is tezepelumab, omalizumab, benzalizumab, mepolizumab, or reslizumab.

78. The method of claim 77, wherein the biological or pharmaceutical drug is tezepelumab.

79. The method of claim 70, wherein the inflammatory disease or disorder is rheumatoid arthritis, psoriatic arthritis, plaque psoriasis, or ankylosing spondylitis.

80. The method of claim 70 or 79, wherein the biological or pharmaceutical drug is etanercept, adalimumab, dupilumab, ustekinumab, infliximab, golimumab, or certolizumab pegol.

81. The method of claim 70, 79, or 80, wherein the biological or pharmaceutical drug is etanercept.

82. The method of any one of the preceding claims, wherein the subject is a human subject.

83. A kit comprising reagents for measuring a level a protein in a biological sample from a subject, wherein the protein is
(a) growth/differentiation factor 15 (GDF15);
(b) thrombospondin-2 (TSP-2);
(c) retinoblastoma-like protein 2 (RBL2);
(d) WAP four-disulfide core domain protein 2 (WFDC2);
(e) anthrax toxin receptor 2 (ANTRX2);
(f) alpha-1-antichymotrypsin complex (ACT COMPLEX or SERPINA3);
(g) spondin-1 (SPON1);
(h) spondin-2 (SPON2);
(i) serum amyloid A-1 protein (SAA1);
(j) serum amyloid A-2 protein (SAA2);
(k) C5a anaphylatoxin (C5aAT);
(l) tumor necrosis factor receptor superfamily member 1A (TNFRSFiA);
(m) angiopoietin-2 (ANG2);
(n) macrophage metalloelastase (MMP12);
(o) transgelin (TAGLN);
(p) sushi, von Willebrand factor type A, EGF and pentraxin domain-containing protein 1 (SVEP1);
(q) hematopoietic prostaglandin D synthase (HPGDS);
(r) disintegrin and metalloproteinase domain-containing protein 22 (ADAM22);
(s) growth hormone receptor (GHR);
(t) insulin-like growth factor-binding protein 2 (IGFPB2);
(u)N-terminal pro-BNP (NTproBNP);
(v) transmembrane emp24 domain-containing protein 10 (TMED10);
(w) pancreatic ribonuclease (RNase1);
(x) cardiac troponin T (cTnT);
(y) cartilage intermediate layer protein 2 (CILP2);
(z) tyrosine-protein kinase transmembrane receptor ROR2 (ROR2);
(aa) beta-2-microglobulin (beta-2-M);
(ab) insulin-like growth factor-binding protein 6 (IGFPB6);
(ac) tetranectin (TN);
(ad) dCTP pyrophosphatase 1 (DCTPP1);
(ae) tumor necrosis factor receptor superfamily member EDAR (EDAR);
(af) erythropoietin (EPO);
(ag) complement component C7 (C7);
(ah) L-Selectin (CD62L);
(ai) netrin receptor UNC5B (UNCB5);
(aj) brorin;
(ak) epidermal growth factor receptor (EGFR);
(al) serine protease inhibitor Kazal-type 5 (SPINK5);
(am) immunoglobulin superfamily member 3 (IGSF3);
(an) Fc receptor-like protein 1 (FCRL1);
(ao) pleiotrophin (PTN);
(ap) RGM domain family member B (RGMB);
(aq) ephrin type-B receptor 2 (EPHB2);
(ar) triggering receptor expressed on myeloid cells 1 (TREM-1);
(as) elafin; or
(at) WNT1-inducible-signaling pathway protein 2 (WISP-2).

84. The kit of claim 83, comprising a means for measuring the level of each of any two or more of the proteins of (a)-(at).

85. The kit of claim 83 or 84, further comprising a means for comparing the measured level of the protein or proteins in the biological sample with a reference level of the protein or proteins.

86. The kit of any one of claims 83-85, further comprising an instruction protocol for measuring and/or comparing the level of the protein or proteins.
